SEQUENCE LISTING (96 sequences)

SEQ ID NO: 1 - protein, 12 aa, Artificial Sequence
  Synthetic construct
CASRVGIAEAFF

SEQ ID NO: 2 - protein, 13 aa, Artificial Sequence
  Synthetic construct
CASSEDSNQPQHF

SEQ ID NO: 3 - protein, 14 aa, Artificial Sequence
  Synthetic construct
CASSLGTGYSPLHF

SEQ ID NO: 4 - protein, 16 aa, Artificial Sequence
  Synthetic construct
CASSEHRGRGNQPQHF

SEQ ID NO: 5 - protein, 11 aa, Artificial Sequence
  Synthetic construct
CATSNRGIQYF

SEQ ID NO: 6 - protein, 15 aa, Artificial Sequence
  Synthetic construct
CASSLGDSIYNEQFF

SEQ ID NO: 7 - protein, 14 aa, Artificial Sequence
  Synthetic construct
CASSSGEANYGYTF

SEQ ID NO: 8 - protein, 15 aa, Artificial Sequence
  Synthetic construct
CASSEWVGGNSPLHF

SEQ ID NO: 9 - protein, 12 aa, Artificial Sequence
  Synthetic construct
CASSQESYEQYF

SEQ ID NO: 10 - protein, 15 aa, Artificial Sequence
  Synthetic construct
CASSRDIGLSQPQHF

SEQ ID NO: 11 - protein, 15 aa, Artificial Sequence
  Synthetic construct
CASSESRGVNGELFF

SEQ ID NO: 12 - protein, 20 aa, Artificial Sequence
  Synthetic construct
CASSIGGGTSGRAGYNEQFF

SEQ ID NO: 13 - protein, 12 aa, Artificial Sequence
  Synthetic construct
CSAQGPHYGYTF

SEQ ID NO: 14 - protein, 16 aa, Artificial Sequence
  Synthetic construct
CASSPPRDYSGNTIYF

SEQ ID NO: 15 - protein, 13 aa, Artificial Sequence
  Synthetic construct
CASSRNRNTEAFF

SEQ ID NO: 16 - protein, 17 aa, Artificial Sequence
  Synthetic construct
CASSVEGGLGSEQPQHF

SEQ ID NO: 17 - protein, 14 aa, Artificial Sequence
  Synthetic construct
CASTQGGRGGEQYF

SEQ ID NO: 18 - protein, 16 aa, Artificial Sequence
  Synthetic construct
CSASIRTADRAEKLFF

SEQ ID NO: 19 - protein, 10 aa, Artificial Sequence
  Synthetic construct
DEGGWACLVY

SEQ ID NO: 20 - protein, 9 aa, Artificial Sequence
  Synthetic construct
MADQLVAVI

SEQ ID NO: 21 - protein, 10 aa, Artificial Sequence
  Synthetic construct
VLYSNRFAAY

SEQ ID NO: 22 - protein, 10 aa, Artificial Sequence
  Synthetic construct
YSNRFAAYAK

SEQ ID NO: 23 - protein, 9 aa, Artificial Sequence
  Synthetic construct
SATMSGVTI

SEQ ID NO: 24 - protein, 10 aa, Artificial Sequence
  Synthetic construct
STPICSSRRK

SEQ ID NO: 25 - protein, 9 aa, Artificial Sequence
  Synthetic construct
EEVLHTMPI

SEQ ID NO: 26 - protein, 9 aa, Artificial Sequence
  Synthetic construct
SISSGESIK

SEQ ID NO: 27 - protein, 11 aa, Artificial Sequence
  Synthetic construct
LVYKEKLIIWK

SEQ ID NO: 28 - protein, 9 aa, Artificial Sequence
  Synthetic construct
GSQVRYACK

SEQ ID NO: 29 - protein, 9 aa, Artificial Sequence
  Synthetic construct
LEDNPESTV

SEQ ID NO: 30 - protein, 9 aa, Artificial Sequence
  Synthetic construct
SIKVLGTEK

SEQ ID NO: 31 - protein, 10 aa, Artificial Sequence
  Synthetic construct
KESQPALELK

SEQ ID NO: 32 - protein, 9 aa, Artificial Sequence
  Synthetic construct
KAHLIRPRK

SEQ ID NO: 33 - protein, 9 aa, Artificial Sequence
  Synthetic construct
YVMASVASV

SEQ ID NO: 34 - protein, 9 aa, Artificial Sequence
  Synthetic construct
DEAYVMASV

SEQ ID NO: 35 - protein, 10 aa, Artificial Sequence
  Synthetic construct
KEILDEAYVM

SEQ ID NO: 36 - protein, 10 aa, Artificial Sequence
  Synthetic construct
SSQPSPSDPK

SEQ ID NO: 37 - protein, 9 aa, Artificial Sequence
  Synthetic construct
SQAAVGPQK

SEQ ID NO: 38 - protein, 10 aa, Artificial Sequence
  Synthetic construct
YLSFIKILLK

SEQ ID NO: 39 - protein, 16 aa, Artificial Sequence
  Synthetic construct
CASSDTARGRNYGYTF

SEQ ID NO: 40 - protein, 12 aa, Artificial Sequence
  Synthetic construct
CASSVVGGELFF

SEQ ID NO: 41 - protein, 17 aa, Artificial Sequence
  Synthetic construct
CASSPYGTESGANVLTF

SEQ ID NO: 42 - protein, 16 aa, Artificial Sequence
  Synthetic construct
CASSQDFRDRAGELFF

SEQ ID NO: 43 - protein, 15 aa, Artificial Sequence
  Synthetic construct
CASSQDPSGSYEQYF

SEQ ID NO: 44 - protein, 12 aa, Artificial Sequence
  Synthetic construct
CASSQEVGGYTF

SEQ ID NO: 45 - protein, 14 aa, Artificial Sequence
  Synthetic construct
CASSQVPGSYEQYF

SEQ ID NO: 46 - protein, 14 aa, Artificial Sequence
  Synthetic construct
CSATGTKTNYGYTF

SEQ ID NO: 47 - protein, 13 aa, Artificial Sequence
  Synthetic construct
CASSFPAYNEQFF

SEQ ID NO: 48 - protein, 15 aa, Artificial Sequence
  Synthetic construct
CSVARVQGASGEQYF

SEQ ID NO: 49 - protein, 13 aa, Artificial Sequence
  Synthetic construct
CAWVPGTSGRLVF

SEQ ID NO: 50 - protein, 14 aa, Artificial Sequence
  Synthetic construct
CASSLGGPSSPLHF

SEQ ID NO: 51 - protein, 15 aa, Artificial Sequence
  Synthetic construct
CASSQLDTYNSPLHF

SEQ ID NO: 52 - protein, 15 aa, Artificial Sequence
  Synthetic construct
CAWSEGQSSGNTIYF

SEQ ID NO: 53 - protein, 13 aa, Artificial Sequence
  Synthetic construct
CASSSQGRAEAFF

SEQ ID NO: 54 - protein, 14 aa, Artificial Sequence
  Synthetic construct
CASSLESLNTEAFF

SEQ ID NO: 55 - protein, 12 aa, Artificial Sequence
  Synthetic construct
CASSPQRDGYTF

SEQ ID NO: 56 - protein, 15 aa, Artificial Sequence
  Synthetic construct
CASSLGGAGTYEQYF

SEQ ID NO: 57 - protein, 14 aa, Artificial Sequence
  Synthetic construct
CASSMDRLYSEAFF

SEQ ID NO: 58 - protein, 13 aa, Artificial Sequence
  Synthetic construct
CSAMPVNTGELFF

SEQ ID NO: 59 - protein, 16 aa, Artificial Sequence
  Synthetic construct
CASSPVLGQVIYGYTF

SEQ ID NO: 60 - protein, 13 aa, Artificial Sequence
  Synthetic construct
CASSIGQNYGYTF

SEQ ID NO: 61 - protein, 16 aa, Artificial Sequence
  Synthetic construct
CASKGYRERSYNEQFF

SEQ ID NO: 62 - protein, 14 aa, Artificial Sequence
  Synthetic construct
CASSYLVGNTEAFF

SEQ ID NO: 63 - protein, 15 aa, Artificial Sequence
  Synthetic construct
CSSVKPQGIGTEAFF

SEQ ID NO: 64 - protein, 17 aa, Artificial Sequence
  Synthetic construct
CASSPWATSGRTDTQYF

SEQ ID NO: 65 - protein, 13 aa, Artificial Sequence
  Synthetic construct
CASSYVGVQPQHF

SEQ ID NO: 66 - protein, 13 aa, Artificial Sequence
  Synthetic construct
CASDGGVSYEQYF

SEQ ID NO: 67 - protein, 14 aa, Artificial Sequence
  Synthetic construct
CASSFDASRNEQFF

SEQ ID NO: 68 - protein, 16 aa, Artificial Sequence
  Synthetic construct
CASSPHDAGADTEAFF

SEQ ID NO: 69 - protein, 14 aa, Artificial Sequence
  Synthetic construct
CASSSFWGYNEQFF

SEQ ID NO: 70 - protein, 13 aa, Artificial Sequence
  Synthetic construct
CATSDTRAQGYTF

SEQ ID NO: 71 - protein, 18 aa, Artificial Sequence
  Synthetic construct
CATSRDLAGAASNQPQHF

SEQ ID NO: 72 - protein, 17 aa, Artificial Sequence
  Synthetic construct
CASSLGLAGVLGETQYF

SEQ ID NO: 73 - protein, 15 aa, Artificial Sequence
  Synthetic construct
CASSEYLAGVTEQFF

SEQ ID NO: 74 - protein, 13 aa, Artificial Sequence
  Synthetic construct
CSASPVLSYEQYF

SEQ ID NO: 75 - protein, 16 aa, Artificial Sequence
  Synthetic construct
CASSLSRMPTNYGYTF

SEQ ID NO: 76 - protein, 16 aa, Artificial Sequence
  Synthetic construct
CASSPPAGGLTDTQYF

SEQ ID NO: 77 - protein, 15 aa, Artificial Sequence
  Synthetic construct
CASSPGTSPYNEQFF

SEQ ID NO: 78 - protein, 16 aa, Artificial Sequence
  Synthetic construct
CASSPDRGSSGNTIYF

SEQ ID NO: 79 - protein, 13 aa, Artificial Sequence
  Synthetic construct
CASSYGLNYGYTF

SEQ ID NO: 80 - protein, 13 aa, Artificial Sequence
  Synthetic construct
CSAKLAPGGELFF

SEQ ID NO: 81 - protein, 16 aa, Artificial Sequence
  Synthetic construct
CSARDNRAGGFAEAFF

SEQ ID NO: 82 - protein, 13 aa, Artificial Sequence
  Synthetic construct
CASGGAHNSPLHF

SEQ ID NO: 83 - protein, 17 aa, Artificial Sequence
  Synthetic construct
CASSGTGYGGPTGELFF

SEQ ID NO: 84 - protein, 14 aa, Artificial Sequence
  Synthetic construct
CASRADRGRNTIYF

SEQ ID NO: 85 - protein, 15 aa, Artificial Sequence
  Synthetic construct
CASRKRTGSTDTQYF

SEQ ID NO: 86 - protein, 14 aa, Artificial Sequence
  Synthetic construct
CASTHWVGNTEAFF

SEQ ID NO: 87 - protein, 16 aa, Artificial Sequence
  Synthetic construct
CASSFTRHRGNEKLFF

SEQ ID NO: 88 - protein, 17 aa, Artificial Sequence
  Synthetic construct
CASSHNREGYSNTEAFF

SEQ ID NO: 89 - protein, 17 aa, Artificial Sequence
  Synthetic construct
CAISWTSGRALINEQYF

SEQ ID NO: 90 - protein, 15 aa, Artificial Sequence
  Synthetic construct
CASSLLLRPNTEAFF

SEQ ID NO: 91 - protein, 14 aa, Artificial Sequence
  Synthetic construct
CASRGGTENQPQHF

SEQ ID NO: 92 - protein, 13 aa, Artificial Sequence
  Synthetic construct
CSVRTGEGQPQHF

SEQ ID NO: 93 - protein, 13 aa, Artificial Sequence
  Synthetic construct
CAWWDAYNSPLHF

SEQ ID NO: 94 - protein, 14 aa, Artificial Sequence
  Synthetic construct
CASSKWGANYGYTF

SEQ ID NO: 95 - protein, 17 aa, Artificial Sequence
  Synthetic construct
CASTTTPNGQGADTQYF

SEQ ID NO: 96 - protein, 14 aa, Artificial Sequence
  Synthetic construct
CAIRITTGDYGYTF

(19) United States
(12) Patent Application Publication
(10) Pub. No.: US 2023/0235008 A1
(43) Pub. Date: Jul. 27, 2023

(54) NOVEL T CELL RECEPTORS (TCRS) THAT REACT TO NEOANTIGENS

(71) Applicant: H. LEE MOFFITT CANCER CENTER AND RESEARCH INSTITUTE, INC., Tampa, FL (US)

(72) Inventors: Eric B. HAURA, St. Pete Beach, FL (US); Benjamin C. CREELAN, Tampa, FL (US); Chao WANG, Tampa, FL (US); Scott ANTONIA, Durham, NC (US)

(21) Appl. No.: 17/799,503

(22) PCT Filed: Feb. 16, 2021
(86) PCT No.: PCT/US2021/018224

§ 371 Date: Aug. 12, 2022

Related U.S. Application Data
(60) Provisional application No. 62/976,867, filed on Feb. 14, 2020.
(60) Provisional application No. 62/978,230, filed on Feb. 18, 2020.
(60) Provisional application No. 63/048,090, filed on Jul. 4, 2020.

(51) Int. Cl. C07K 14/725; A61P 35/00; A61K 35/17; C12N 5/0783; A61K 31/4545; A61K 31/506; A61K 38/17

(52) U.S. Cl. CPC C07K 14/7051; A61P 35/00; A61K 35/17; C12N 5/0636; A61K 31/4545; A61K 31/506; A61K 38/1774; C07K 2317/24

(57) ABSTRACT

Disclosed are T cell receptors (TCRs) specific for one or more neoantigens and T cells engineered to express said TCRs as well as methods of their use for treating cancer.

This application claims the benefit of U.S. Provisional Application No. 63/048,090, filed on Jul. 4, 2020, U.S. Provisional Application No. 62/978,230, filed on Feb. 18, 2020, and U.S. Provisional Application No. 62/976,867, filed on Feb. 14, 2020, application s which are incorporated herein by reference in their entirety.

### I. BACKGROUND

Neoantigens are antigens created by non-synonymous somatic mutations and recognized by unique T cell receptor (TCR) clonotypes of CD8 or CD4. Studies into neoantigens have shown that some neoantigens that have been able to be identified may induce the durable remissions >1 decade via adoptive cell transfer. Once identified as a target, a neoantigen alone is not necessarily the end of the therapeutic discovery as TCRs can be designed that recognize neoantigens. Thus, what are needed are convenient and economic methods to identify relevant neoantigens for clinical applications is urgently needed.

### II. SUMMARY

Disclosed are TCRs that recognize one or more neoantigens and methods of their use.

In one aspect, disclosed herein are T cell receptors that recognizes one or more neoantigens including, but not limited to DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), YLSFIKILLK (SEQ ID NO: 38), ASPM with a 11250S substitution, NUP133 with a C119Y substitution, ABCC2 with a G664D substitution, ABCC3 with a Y1298C substitution, ABCA12 with a R898I substitution, GPD2 with a D423Y substitution, TP53 with a T155N substitution, MEX3D with a E477Q substitution, SMAD4 with a D537E substitution, TUBB with a T238I substitution, BIRC6 with a V1860I substitution, STIP with a S45F substitution, MAGE-A1, MAGE-A4, MAGE-A5, MAGE-A6, TMPRSS11F with a S306L substitution, and/or any of the neoantigens recited in Table 1.

Also disclosed herein are TCRs of any preceding aspect, wherein the TCR comprises the sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), CASSDTARGRNYGYTF (SEQ ID NO: 39), CASSVVGGELFF (SEQ ID NO: 40), CASSPYGTESGANVLTF (SEQ ID NO: 41), CASSQDFRDRAGELFF (SEQ ID NO: 42), CASSQDPSGSYEQYF (SEQ ID NO: 43), CASSQEVGGYTF (SEQ ID NO: 44), CASSQVPGSYEQYF (SEQ ID NO: 45), CSATGTKTNYGYTF (SEQ ID NO: 46), CASSFPAYNEQFF (SEQ ID NO: 47), CSVARVQGASGEQYF (SEQ ID NO: 48), CAWVPGTSGRLVF (SEQ ID NO: 49), CASSLGGPSSPLHF (SEQ ID NO: 50), CASSQLDTYNSPLHF (SEQ ID NO: 51), CAWSEGQSSGNTIYF (SEQ ID NO: 52), CASSSQGRAEAFF (SEQ ID NO: 53), CASSLESLNTEAFF (SEQ ID NO: 54), CASSPQRDGYTF (SEQ ID NO: 55), CASSLGGAGTYEQYF (SEQ ID NO: 56), CASSMDRLYSEAFF (SEQ ID NO: 57), CSAMPVNTGELFF (SEQ ID NO: 58), CASSPVLGQVIYGYTF (SEQ ID NO: 59), CASSIGQNYGYTF (SEQ ID NO: 60), CASKGYRERSYNEQFF (SEQ ID NO: 61), CASSYLVGNTEAFF (SEQ ID NO: 62), CSSVKPQGIGTEAFF (SEQ ID NO: 63), CASSPWATSGRTDTQYF (SEQ ID NO: 64), CASSYVGVQPQHF (SEQ ID NO: 65), CASDGGVSYEQYF (SEQ ID NO: 66), CASSFDASRNEQFF (SEQ ID NO: 67), CASSPHDAGADTEAFF (SEQ ID NO: 68), CASSSFWGYNEQFF (SEQ ID NO: 69), CATSDTRAQGYTF (SEQ ID NO: 70), CATSRDLAGAASNQPQHF (SEQ ID NO: 71), CASSLGLAGVLGETQYF (SEQ ID NO: 72), CASSEYLAGVTEQFF (SEQ ID NO: 73), CSASPVLSYEQYF (SEQ ID NO: 74), CASSLSRMPTNYGYTF (SEQ ID NO: 75), CASSPPAGGLTDTQYF (SEQ ID NO: 76), CASSPGTSPYNEQFF (SEQ ID NO: 77), CASSPDRGSSGNTIYF (SEQ ID NO: 78), CASSYGLNYGYTF (SEQ ID NO: 79), CSAKLAPGGELFF (SEQ ID NO: 70), CSARDNRAGGFAEAFF (SEQ ID NO: 81), CASGGAHNSPLHF (SEQ ID NO: 82), CASSGTGYGGPTGELFF (SEQ ID NO: 83), CASRADRGRNTIYF (SEQ ID NO: 84), CASRKRTGSTDTQYF (SEQ ID NO: 85), CASTHWVGNTEAFF (SEQ ID NO: 86), CASSFTRHRGNEKLFF (SEQ ID NO: 87), CASSHNREGYSNTEAFF (SEQ ID NO: 88), CAISWTSGRALINEQYF (SEQ ID NO: 89), CASSLLLRPNTEAFF (SEQ ID NO: 90), CASRGGTENQPQHF (SEQ ID NO: 91), CSVRTGEGQPQHF (SEQ ID NO: 92), CAWWDAYNSPLHF (SEQ ID NO: 93), CASSKWGANYGYTF (SEQ ID NO: 94), CASTTTPNGQGADTQYF (SEQ ID NO: 95), or CAIRITTGDYGYTF (SEQ ID NO: 96).

In one aspect, disclosed herein are T cells (such as, for example, a tumor infiltrating lymphocyte (TIL), chimeric antigen receptor (CAR) T cell, or marrow infiltrating lymphocyte (MIL)) comprising the TCR of any preceding aspect.

Also disclosed herein are methods of treating, inhibiting, reducing, decreasing, ameliorating, and/or preventing a cancer and/or metastasis (such as, for example, a tyrosine kinase inhibitor resistant or EGFR mutated cancer) in a subject comprising administering to the subject a TCR, T cell, CAR T cell, TIL, and/or MIL of any preceding aspect. For example disclosed herein are methods of treating a cancer in a subject comprising administering to the subject a T cell, CAR T cell, TIL, and/or MIL comprising a TCR that recognizes a neoantigen including, but not limited to, neoantigens comprising the sequence DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), YLSFIKILLK (SEQ ID NO: 38), ASPM with a 11250S substitution, NUP133 with a C119Y substitution, ABCC2 with a G664D substitution, ABCC3 with a Y1298C substitution, ABCA12 with a R898I substitution, GPD2 with a D423Y substitution, TP53 with a T155N substitution, MEX3D with a E477Q substitution, SMAD4 with a D537E substitution, TUBB with a T238I substitution, BIRC6 with a V1860I substitution, STIP with a S45F substitution, MAGE-A1, MAGE-A4, MAGE-A5, MAGE-A6, TMPRSS11F with a S306L substitution, and/or any of the neoantigens recited in Table 1. In one aspect, disclosed herein are methods of treating, inhibiting, reducing, decreasing, ameliorating, and/or preventing a cancer and/or metastasis in a subject of any preceding aspect, wherein the TCR comprises the sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), CASSDTARGRNYGYTF (SEQ ID NO: 39), CASSVVGGELFF (SEQ ID NO: 40), CASSPYGTESGANVLTF (SEQ ID NO: 41), CASSQDFRDRAGELFF (SEQ ID NO: 42), CASSQDPSGSYEQYF (SEQ ID NO: 43), CASSQEVGGYTF (SEQ ID NO: 44), CASSQVPGSYEQYF (SEQ ID NO: 45), CSATGTKTNYGYTF (SEQ ID NO: 46), CASSFPAYNEQFF (SEQ ID NO: 47), CSVARVQGASGEQYF (SEQ ID NO: 48), CAWVPGTSGRLVF (SEQ ID NO: 49), CASSLGGPSSPLHF (SEQ ID NO: 50), CASSQLDTYNSPLHF (SEQ ID NO: 51), CAWSEGQSSGNTIYF (SEQ ID NO: 52), CASSSQGRAEAFF (SEQ ID NO: 53), CASSLESLNTEAFF (SEQ ID NO: 54), CASSPQRDGYTF (SEQ ID NO: 55), CASSLGGAGTYEQYF (SEQ ID NO: 56), CASSMDRLYSEAFF (SEQ ID NO: 57), CSAMPVNTGELFF (SEQ ID NO: 58), CASSPVLGQVIYGYTF (SEQ ID NO: 59), CASSIGQNYGYTF (SEQ ID NO: 60), CASKGYRERSYNEQFF (SEQ ID NO: 61), CASSYLVGNTEAFF (SEQ ID NO: 62), CSSVKPQGIGTEAFF (SEQ ID NO: 63), CASSPWATSGRTDTQYF (SEQ ID NO: 64), CASSYVGVQPQHF (SEQ ID NO: 65), CASDGGVSYEQYF (SEQ ID NO: 66), CASSFDASRNEQFF (SEQ ID NO: 67), CASSPHDAGADTEAFF (SEQ ID NO: 68), CASSSFWGYNEQFF (SEQ ID NO: 69), CATSDTRAQGYTF (SEQ ID NO: 70), CATSRDLAGAASNQPQHF (SEQ ID NO: 71), CASSLGLAGVLGETQYF (SEQ ID NO: 72), CASSEYLAGVTEQFF (SEQ ID NO: 73), CSASPVLSYEQYF (SEQ ID NO: 74), CASSLSRMPTNYGYTF (SEQ ID NO: 75), CASSPPAGGLTDTQYF (SEQ ID NO: 76), CASSPGTSPYNEQFF (SEQ ID NO: 77), CASSPDRGSSGNTIYF (SEQ ID NO: 78), CASSYGLNYGYTF (SEQ ID NO: 79), CSAKLAPGGELFF (SEQ ID NO: 70), CSARDNRAGGFAEAFF (SEQ ID NO: 81), CASGGAHNSPLHF (SEQ ID NO: 82), CASSGTGYGGPTGELFF (SEQ ID NO: 83), CASRADRGRNTIYF (SEQ ID NO: 84), CASRKRTGSTDTQYF (SEQ ID NO: 85), CASTHWVGNTEAFF (SEQ ID NO: 86), CASSFTRHRGNEKLFF (SEQ ID NO: 87), CASSHNREGYSNTEAFF (SEQ ID NO: 88), CAISWTSGRALINEQYF (SEQ ID NO: 89), CASSLLLRPNTEAFF (SEQ ID NO: 90), CASRGGTENQPQHF (SEQ ID NO: 91), CSVRTGEGQPQHF (SEQ ID NO: 92), CAWWDAYNSPLHF (SEQ ID NO: 93), CASSKWGANYGYTF (SEQ ID NO: 94), CASTTTPNGQGADTQYF (SEQ ID NO: 95), or CAIRITTGDYGYTF (SEQ ID NO: 96).

Also disclosed herein are methods of treating, inhibiting, reducing, decreasing, ameliorating, and/or preventing a cancer and/or metastasis in a subject of any preceding aspect, wherein the TILs, MILs, T cells, and/or CAR T cells are expanded in vitro in the presence of one or more of the neoantigens prior to administration of the TILs.

In one aspect, disclosed herein are methods of treating, inhibiting, reducing, decreasing, ameliorating, and/or preventing a cancer and/or metastasis in a subject of any preceding aspect, further comprising administering to the subject the neoantigen which the T cell, CAR T cell, TIL, and/or MIL recognizes. In some aspects, the TILs, MILs, CAR T cell, or T cell and neoantigen are administered in the same formulation or concurrently. When administered separately, the TILs, MILs, CAR T cell, or T cells and neoantigen can be administered concurrently or 1, 2, 3, 4, 5 6, 7, 8, 9, 10, 11, 12, 13, 14, 15, 20, 25, 30, 35, 40, 45, 50, 55, 60, 65, 70, 75, 80, 85, 90, 120 min, 3, 4, 5, 6, 7, 8, 9, 10, 11, 12, 13, 14, 15, 16, 17, 18, 19, 20, 21, 22, 23, 24, 28, 30, 36, 42, 48 hours, 3, 4, 5, 6, 7, 8, 9, 10, 11, 12, 13, 14 days apart with either administration preceding the other.

Also disclosed herein are methods of treating, inhibiting, reducing, decreasing, ameliorating, and/or preventing a cancer and/or metastasis in a subject of any preceding aspect, wherein the T cells, CAR T cells, TILs, and/or MILs are obtained from the subject that is being treated.

In one aspect, disclosed herein are methods of treating, inhibiting, reducing, decreasing, ameliorating, and/or preventing a cancer and/or metastasis in a subject of any preceding aspect further comprising the administration of an anti-cancer therapeutic agent.

In one aspect, disclosed herein are methods of treating, inhibiting, reducing, decreasing, ameliorating, and/or preventing a cancer and/or metastasis in a subject of any preceding aspect further comprising administering to the subject an immune checkpoint inhibitor (such as, for example, a PD1/PDL1 blockade inhibitors and/or CTLA4/B7-1 or 2 inhibitors (such as, for example, PD-1 inhibitors lambrolizumab, OPDIVO® (Nivolumab), KEYTRUDA® (pembrolizumab), and pidilizumab; PD-L1 inhibitors BMS-936559, TECENTRIQ® (Atezolizumab), IMFINZI® (Durvalumab), and BAVENCIO® (Avelumab); and CTLA-4 inhibitors YERVOY (ipilimumab)).

Also disclosed herein are methods of treating, inhibiting, reducing, decreasing, ameliorating, and/or preventing a cancer and/or metastasis in a subject of any preceding aspect further comprising administering to the subject cancer crizotinib, ceritinib, alectinib, brigatinib, vemurafenib, dabrafenib, afatinib, Tivantinib, AMG 102, ficlatuzumab, cabozantinib, foretinib, ponatinib, onartuzumab, LKD378, AP26113, TSR-011, Selumetinib, TAE684, Trametinib, barbozatinib, gefitinib, erlotinib, paptinib, vandetanib, afatinib, osimertinib, lenvatinib, nintedanib, pazopanib, regorafenib, sorafenib, sunitinib, bosutinib, dasatinib, imatinib, nilotinib, and/or ponatinib.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a schematic representation of a neoantigen identification flowchart for a peptide-based screening method to identify neoantigen in NSCLC patients.

FIG. 2 shows a schematic for TCRVβ sequencing.

FIG. 3 shows identification and expansion of neotantigen specific TCRVβ clonotypes.

FIG. 4 shows MANAFEST+ data for various clonotypes.

FIG. 5 shows all MANAFEST+ TCRVβ clonotypes.

FIG. 6 shows the tracking of TCRVβ after TIL infusion.

FIG. 7 shows persistence of infused TRVβ clonotypes at week 16 and the correlation with benefit. Shown is the mean+/−standard error for Morisita's overlap index for TCR VP chain productive rearrangement amino acid sequences between final infused TIL product and peripheral blood mononuclear cells at indicated timepoints. “Clinical benefit” denotes >12 months of clinical remission without requiring additional systemic therapy. Representative patient group (n=10), final sample-set under analysis and still accruing.

FIG. 8 shows dynamics of neoantigen-specific T cells over time.

FIGS. 9A and 9B show trial schematic and patient disposition. FIG. 9A shows clinical trial schema. Day count is relative to TIL infusion. FIG. 9B shows patient disposition per Consolidated Standards of Reporting Trials (CONSORT) guidelines. Abbreviations: TKI denotes tyrosine kinase inhibitor; IL-2, interleukin-2; TIL, tumor-infiltrating lymphocyte treatment; PD, progressive disease; q w, every week; ID, patient number.

FIGS. 10A and 10B show clinical follow-up after initial nivolumab. FIG. 10A show the sum of tumor diameters per RECIST v1.1 over time measured by serial CT scan. Patients with evidence of tumor enlargement or new lesions within 10 weeks of initial nivolumab were treated with TIL infusion. All enrolled patients (n=20) are included. FIG. 10B show clinical follow-up of patients after initial nivolumab with clinicopathologic features in a Swimmer's plot. Pt 04 received bridging crizotinib due to rapid progression on nivolumab. Abbreviations: TPS denotes tumor proportion score using 22C3 PD-L1 antibody. TMB; estimated tumor mutation burden based upon exome sequencing. mut/MB mutations per mega-base. ‘Ex’ denotes exon, and ‘A’ denotes mutation conferring exon deletion. Smoking pack-years represents self-reported cigarette packs per day multiplied by total years.

FIGS. 11A and 11B show the feasibility of TIL harvest and intermediate-dose interleukin-2 infusion for all patients. FIG. 11A shows the total hours of length of stay (LOS) for inpatient admissions after TIL harvest surgery. Two patients had postoperative air-leak requiring multiple days of inpatient observation. All enrolled patients (n=20) are included. FIG. 11B shows the total duration of infusional IL-2 for all 16 patients after TIL treatment. Patients received TIL infusion on Day 0, followed by continuous IL-2 infusion beginning 12 hours later at a dose of 18 million international units (MIU) per m2 over 6, then 12, and then 24 hours followed by 4.5 MIU/m2 over 24 hours for 3 consecutive days.

FIG. 12 shows a waterfall plot showing best overall change in sum of diameter of tumor lesions. Change in sum of tumor diameters by RECIST is compared to Week-1 prior to TIL. Pt 14, designated as radiographic “PD” post-TIL, had biopsy showing of only target lesion showing fibrosis with no tumor cells. All patients evaluable with a post-TIL CT scan are included (n=13). “*” denotes initial PR with nivolumab, followed by biopsy-proven unequivocal PD (ID 07, 09), ‡ Unconfirmed PR, subsequent non-target PD (ID 02, 05, 31); §§ Had 10 mm increase in target lesion on prior nivolumab. (ID 08).

FIGS. 13A, 13B, and 13C show the clinical activity of TIL and patient survival. FIG. 13A shows features of patients who were treated with TIL and their clinical outcomes (n=16). FIG. 13B shows the change in sum of tumor diameters, relative to Week-1 prior to TIL. Pt 14, designated as radiographic “PD” post-TIL, had biopsy of only target lesion showing fibrosis with no tumor cells at the time of progression. FIG. 13C shows overall survival of all treated patients from date of TIL infusion. Hashmarks denote 95% confidence intervals. Median follow-up of 1.6 years (range 0.5-2.9) by reverse Kaplan-Meier method. Abbreviations: TPS denotes tumor proportion score by 22C3 antibody immunohistochemistry; IL-2, interleukin-2 and TIL, tumor-infiltrating lymphocyte treatment. TMB, estimated tumor mutation burden; mut/MB, mutations per mega-base. ‘Ex’ denotes exon, and A denotes exon deletion. Smoking pack-years represents self-reported cigarette packs per day multiplied by total years.

FIG. 14 shows a radiographic enlargement of target lesion in a patient 14. Patient had biopsy-proven lung adenocarcinoma biopsy proven from a lymph node metastasis and pleural mass. She had increase in her only target lesion pleural metastasis post-TIL and biopsy revealed fibrosis only. Whole exome sequencing conducted on the original tumor showed over four hundred somatic mutations. Only 3 mutations were detected in the post-TIL biopsy despite 200× depth of sequencing. She had new metastases appear 1.4 years later.

FIG. 15 shows the overall survival from enrollment. Overall survival of all enrolled patients (n=20) from date of enrollment, with hashmark for 95% confidence intervals. All patients were assessed for survival through the data cut-off of 10 Sep. 2020.

FIGS. 16A, 16B, 16C, 16D, 16E, and 16F show examples of complete responses mediated by TIL recognizing tumor antigens. FIG. 16A shows a patient (ID 25) with an EGFR^{ΔEx19} tumor had progressive metastases with nivolumab, followed by complete response to TIL. The sum of radiographic target lesions is shown over time with representative contrast-enhanced axial CT images. FIG. 16B shows IFN-γ spot formation after co-culture of her post-TIL T cells with her autologous dendritic cells pulsed with synthesized long peptides displaying tumor antigens. Five proteins are shown, of 93 tested, mean±SEM of 3 plates over 2 experiments. FIG. 16C shows absolute number of antigen-specific T cell receptor clonotypes within the peripheral blood after TIL. The absolute number and proportion increased after TIL, and then gradually decayed. Data is derived from a total of 10 serial blood samples. The proportion of antigen-specific clones in her infused TIL product is shown in the pie chart. FIG. 16D shows that patient (ID 09) is a former smoker with lung adenocarcinoma. She partial response to nivolumab followed by enlargement of metastatic lymph nodes and a new biopsy-proven rib soft tissue metastasis 10 months later. She was then treated with TIL and had a complete response with ongoing absence of radiographic target lesions. Representative coronal contrast-enhanced CT images are shown over time. FIG. 16E shows co-culture of either CD4⁺ or CD8⁺ T cells sorted from the final infused TIL with autologous dendritic cells and custom peptides corresponding to tumor neoantigens elicited reactivity. Three positive peptides are shown, of 64 tested. Mean±SEM of 3 plates over two experiments. FIG. 16F shows absolute number of antigen-specific clonotypes in the peripheral blood increased and then gradually decayed after the TIL infusion. Data is derived from a total of 13 serial blood samples. Abbreviations: TRVβ, T cell receptor variable beta chain; MAGE, melanoma associated gene; IFN, interferon; Pt, patient.

FIGS. 17A and 17B show In vitro expansion of autologous T-cell clonotypes after stimulation with peptide antigens. Clonotypes with significant increase using autologous T and dendritic cells from 17A, Patient 25 and 17B, Patient 9. T cells were co-cultured with autologous dendritic cells for 10 days and compared to controls wells including T cells only and no peptides. TCR CDR3 AA denotes T-cell receptor VP complementarity-determining region 3 amino acid. Fold expansion is relative to baseline control without peptide.

FIG. 18 shows a summary of tumor-specific antigens tested for all patients. Top panel shows the total sum of unique genomic alterations tested for each patient. Bottom panel shows the tumor-specific antigens which were detected by autologous T cells. Positive antigens were assessed for T cell reactivity by synthesis of the corresponding recombinant peptides, and identification of IFN-γ colony formation by ELISpot, relative to controls with autologous T cells and dendritic cells only. Mean spot forming units per positive Ag are shown, with n=2-5 tests per antigen. Cultured TIL was used to assess antigen reactivity for all patients except 02, 25, 32, 33 due to high IFN-γ background of the cultured TIL. In these instances, autologous T cells isolated from post-TIL peripheral blood were used. Abbreviations: IFN, interferon; Pt, patient; TIL, tumor-infiltrating lymphocyte; Ag, antigen.

FIGS. 19A, 19B, 19C and 19D show the change in phenotype and genotype of peripheral T cells after TIL infusion. FIG. 19A shows the increase in circulating subsets of polyfunctional CD8⁺ T cells at post-infusion timepoints. *Mixed-effects model (REML) with stacked matching with Geisser-Greenhouse correction. Multiplicity adjusted p value using Dunnett's control. Available dataset with 12 tested patients. FIG. 19B shows the increase in peripheral CD45RA⁻CCR7⁻ effector memory T cells at post-infusion timepoints for all patients with available timepoints (n=15) with 8 serial timepoints collected per patient. Stacked area curves denote mean±SEM. FIG. 19C shows the overlap of TRW chain productive rearrangements between final infused TIL product and peripheral blood T cells at indicated time-points, using Morasita's index. Bars denote mean±SEM of all patients with available timepoints for analysis (n=16). Abbreviations: TRW, T cell receptor variable beta chain; SEM, standard error; TIL, tumor-infiltrating lymphocyte infusion; nivo, nivolumab; MIP, macrophage inflammatory protein; IFN, interferon. REML, restricted (or residual, or reduced) maximum likelihood; CCR, chemokine receptor.

FIGS. 20A and 20B show no association of phenotypic features of infused TIL and overall response. FIG. 20A shows the best overall response (BORR) and proportion of tumor-specific fragments in the final product. All patients with sufficient TIL for autologous reactivity testing performance are shown (n=18). Pt 26 had insufficient tumor digest for autologous reactivity testing, and Pt 12 had insufficient pre-REP TIL for testing. FIG. 20B shows patient BORR and dose of CD8 or CD4 cells in final expanded TIL product. All patients with final manufactured TIL are shown (n=18). Pt 30 pre-REP TIL is still cryopreserved, and Pt 12 had insufficient pre-REP TIL to manufacture. 48 minced tumor fragments were cultured in IL-2. A proportion were tested for reactivity with autologous tumor suspension using ELISA for interferon-γ. Pink is the proportion of autologous-reactively cultures pooled for the final rapid expansion. Gray are either non-specific or negative cultures added to the pool, to ensure sufficient starting cell numbers. Abbreviations: NT; not treated. NE; not evaluable. uPR, unconfirmed partial response.

FIGS. 21A and 21B show treatment-emergent adverse events reported with cyclophosphamide, fludarabine, TIL, and IL-2. FIG. 21A shows adverse events grouped by National Cancer Institute preferred term occurring in 20% or more of patients attributable to cyclophosphamide, fludarabine, TIL, or IL-2. All TIL treated patients (n=16) are included. FIG. 21B shows all adverse events grouped by date of onset within 3 months of TIL. Patients with multiple events for a given term are counted once, using the maximum grade under each preferred term. Abbreviations: AE denotes adverse events; AST denotes aspartate aminotransferase; ALT denotes alanine aminotransferase; IL-2, interleukin-2.

FIGS. 22A and 22B show the change in lymphocytes and neutrophils with lymphodepletion, TIL, and IL2. FIG. 22A shows the initial peak followed by recovery of peripheral blood absolute lymphocyte count. FIG. 22B shows peripheral blood absolute neutrophil count recovered by median of 7.5 days (range 4.7-20.6). Shown is absolute cell count (1000 cells per mm3). TIL infusion is “Day 0”. All TIL treated patients are shown (n=16).

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

In this specification and in the claims that follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

A “binding molecule” or “antigen binding molecule” (e.g., an antibody or antigen-binding fragment thereof) as provided herein refers in its broadest sense to a molecule that specifically binds an antigenic determinant In one embodiment, the binding molecule specifically binds to an immunoregulator molecule (such as for example, a transmembrane SEMA4D (CD100) polypeptide of about 150 kDa or a soluble SEMA4D polypeptide of about 120 kDa). In another embodiment, a binding molecule is an antibody or an antigen binding fragment thereof, e.g., MAb 67 or pepinemab.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. T Cells Comprising T Cell Receptors that are Reactive to One or More Neoantigens and Methods of their Use

In one aspect, disclosed herein are methods of screening for neoantigens, the method comprising: a) obtaining a cancerous tissue sample (such as a tissue sample from a metastasis) from a subject with a cancer; b) fragmenting a first portion of the tissue sample and culturing said first portion; c) expanding tumor infiltrating lymphocytes (TILs) in the cultured first portion; d) subjecting a second portion of the tissue sample to sequencing (such as, for example whole exosome sequencing or RNA sequencing); e) applying bioinformatics to the sequence data to identify putative neoantigens; f) co-culturing the putative neoantigens with the expanded TILs; and g) assaying the co-cultured TILs for reactivity to cancer cells from the subject (for example, assaying for reactivity wherein the reactivity is determined by ELISA, ELISpot, and/or TCRVβ sequencing); wherein reactive TILs indicate that the putative neoantigen co-cultured with the TILs is a neoantigen.

The disclosure herein provide for T cell receptors that are specific for one or more neoantigens and can be used for the treatment of a cancer. Accordingly, in one aspect, disclosed herein are T cell receptors that recognizes for one or more neoantigens including, but not limited to DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), YLSFIKILLK (SEQ ID NO: 38), ASPM with a 11250S substitution, NUP133 with a C119Y substitution, ABCC2 with a G664D substitution, ABCC3 with a Y1298C substitution, ABCA12 with a R898I substitution, GPD2 with a D423Y substitution, TP53 with a T155N substitution, MEX3D with a E477Q substitution, SMAD4 with a D537E substitution, TUBB with a T238I substitution, BIRC6 with a V1860I substitution, STIP with a S45F substitution, MAGE-A1, MAGE-A4, MAGE-A5, MAGE-A6, TMPRSS11F with a 5306L substitution, and/or any of the neoantigens recited in Table 1. In one aspect, the TCR can comprise the sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), CASSDTARGRNYGYTF (SEQ ID NO: 39), CASSVVGGELFF (SEQ ID NO: 40), CASSPYGTESGANVLTF (SEQ ID NO: 41), CASSQDFRDRAGELFF (SEQ ID NO: 42), CASSQDPSGSYEQYF (SEQ ID NO: 43), CASSQEVGGYTF (SEQ ID NO: 44), CASSQVPGSYEQYF (SEQ ID NO: 45), CSATGTKTNYGYTF (SEQ ID NO: 46), CASSFPAYNEQFF (SEQ ID NO: 47), CSVARVQGASGEQYF (SEQ ID NO: 48), CAWVPGTSGRLVF (SEQ ID NO: 49), CASSLGGPSSPLHF (SEQ ID NO: 50), CASSQLDTYNSPLHF (SEQ ID NO: 51), CAWSEGQSSGNTIYF (SEQ ID NO: 52), CASSSQGRAEAFF (SEQ ID NO: 53), CASSLESLNTEAFF (SEQ ID NO: 54), CASSPQRDGYTF (SEQ ID NO: 55), CASSLGGAGTYEQYF (SEQ ID NO: 56), CASSMDRLYSEAFF (SEQ ID NO: 57), CSAMPVNTGELFF (SEQ ID NO: 58), CASSPVLGQVIYGYTF (SEQ ID NO: 59), CASSIGQNYGYTF (SEQ ID NO: 60), CASKGYRERSYNEQFF (SEQ ID NO: 61), CASSYLVGNTEAFF (SEQ ID NO: 62), CSSVKPQGIGTEAFF (SEQ ID NO: 63), CASSPWATSGRTDTQYF (SEQ ID NO: 64), CASSYVGVQPQHF (SEQ ID NO: 65), CASDGGVSYEQYF (SEQ ID NO: 66), CASSFDASRNEQFF (SEQ ID NO: 67), CASSPHDAGADTEAFF (SEQ ID NO: 68), CASSSFWGYNEQFF (SEQ ID NO: 69), CATSDTRAQGYTF (SEQ ID NO: 70), CATSRDLAGAASNQPQHF (SEQ ID NO: 71), CASSLGLAGVLGETQYF (SEQ ID NO: 72), CASSEYLAGVTEQFF (SEQ ID NO: 73), CSASPVLSYEQYF (SEQ ID NO: 74), CASSLSRMPTNYGYTF (SEQ ID NO: 75), CASSPPAGGLTDTQYF (SEQ ID NO: 76), CASSPGTSPYNEQFF (SEQ ID NO: 77), CASSPDRGSSGNTIYF (SEQ ID NO: 78), CASSYGLNYGYTF (SEQ ID NO: 79), CSAKLAPGGELFF (SEQ ID NO: 70), CSARDNRAGGFAEAFF (SEQ ID NO: 81), CASGGAHNSPLHF (SEQ ID NO: 82), CASSGTGYGGPTGELFF (SEQ ID NO: 83), CASRADRGRNTIYF (SEQ ID NO: 84), CASRKRTGSTDTQYF (SEQ ID NO: 85), CASTHWVGNTEAFF (SEQ ID NO: 86), CASSFTRHRGNEKLFF (SEQ ID NO: 87), CASSHNREGYSNTEAFF (SEQ ID NO: 88), CAISWTSGRALINEQYF (SEQ ID NO: 89), CASSLLLRPNTEAFF (SEQ ID NO: 90), CASRGGTENQPQHF (SEQ ID NO: 91), CSVRTGEGQPQHF (SEQ ID NO: 92), CAWWDAYNSPLHF (SEQ ID NO: 93), CASSKWGANYGYTF (SEQ ID NO: 94), CASTTTPNGQGADTQYF (SEQ ID NO: 95), or CAIRITTGDYGYTF (SEQ ID NO: 96).

TABLE 1
Neoantigens

| Mutation | symbol | aa_change | aa_class | mut_25mer(aa) | nrm_25mer(aa) |
|---|---|---|---|---|---|
| SU2C3_chr1_ | ASPM | chr1s | H−>PU | DMSNTIPDEKVVSTYLS | DMSNTIPDEKVVITYL |
| 197091166 | | | | FLCARLLD | SFLCARLLD |
| SU2C3_chr1_ | NUP133 | C−>Y | PU−>H | INIDEGGWACLVYKEKL | INIDEGGWACLVCKEK |
| 229637795 | | | | IIWKIALS | LIIWKIALS |
| SU2C3_chr19_ | MEX3D | E−>Q | A−>PU | TVPAAATIWAPFQRAAP | TVPAAATIWAPFERAA |
| 1556089 | | | | LPAFSGCS | PLPAFSGCS |
| SU2C3_chr18_ | SMAD4 | D−>E | A−>A | IEIHLHRALQLLEEVLH | IEIHLHRALQLLDEVL |
| 48604789 | | | | TMPIADPQ | HTMPIADPQ |
| SU2C3_chr2_ | BIRC6 | V−>I | H−>H | IPPPVCRFMKITIIGRY | IPPPVCRFMKITVIGR |
| 32692977 | | | | GSTNARAK | YGSTNARAK |
| SU2C3_chr1_ | ASPM | I−>S | H−>PU | DMSNTIPDEKVVSTYLS | DMSNTIPDEKVVITYL |
| 262184424 | | | | FLCARLLD | SFLCARLLD |
| SU2C3_chr1_ | NUP134 | C−>Y | PU−>H | INIDEGGWACLVYKEKL | INIDEGGWACLVCKEK |
| 294731053 | | | | IIWKIALS | LIIWKIALS |
| SU2C3_chr19_ | MEX3D | E−>Q | A−>PU | TVPAAATIWAPFQRAAP | TVPAAATIWAPFERAA |
| 1556090 | | | | LPAFSGCS | PLPAFSGCS |
| SU2C3_chr18_ | SMAD5 | D−>E | A−>A | IEIHLHRALQLLEEVLH | IEIHLHRALQLLDEVL |
| 48604790 | | | | TMPIADPQ | HTMPIADPQ |
| SU2C3_chr2_ | BIRC7 | V−>I | H−>H | IPPPVCRFMKITIIGRY | IPPPVCRFMKITVIGR |
| 32692978 | | | | GSTNARAK | YGSTNARAK |
| SU2C3_chr1_ | ASPM | I−>S | H−>PU | DMSNTIPDEKVVSTYLS | DMSNTIPDEKVVITYL |
| 327277682 | | | | FLCARLLD | SFLCARLLD |
| SU2C3_chr1_ | NUP135 | C−>Y | PU−>H | INIDEGGWACLVYKEKL | INIDEGGWACLVCKEK |
| 359824311 | | | | HWKIALS | LHWKIALS |
| SU2C3_chr19_ | MEX3D | E−>Q | A−>PU | TVPAAATIWAPFQRAAP | TVPAAATIWAPFERAA |
| 1556091 | | | | LPAFSGCS | PLPAFSGCS |
| SU2C3_chr18_ | SMAD6 | D−>E | A−>A | IEIHLHRALQLLEEVLH | IEIHLHRALQLLDEVL |
| 48604791 | | | | TMPIADPQ | HTMPIADPQ |
| SU2C3_chr2_ | BIRC8 | V−>I | H−>H | IPPPVCRFMKITIIGRY | IPPPVCRFMKITVIGR |
| 32692979 | | | | GSTNARAK | YGSTNARAK |
| SU2C3_chr1_ | ASPM | I−>S | H−>PU | DMSNTIPDEKVVSTYLS | DMSNTIPDEKVVITYL |
| 392370940 | | | | FLCARLLD | SFLCARLLD |
| SU2C3_chr1_ | NUP136 | C−>Y | PU−>H | INIDEGGWACLVYKEKL | INIDEGGWACLVCKEK |
| 424917569 | | | | HWKIALS | LHWKIALS |
| SU2C3_chr19_ | MEX3D | E−>Q | A−>PU | TVPAAATIWAPFQRAAP | TVPAAATIWAPFERAA |
| 1556092 | | | | LPAFSGCS | PLPAFSGCS |
| SU2C3_chr18_ | SMAD7 | D−>E | A−>A | IEIHLHRALQLLEEVLH | IEIHLHRALQLLDEVL |
| 48604792 | | | | TMPIADPQ | HTMPIADPQ |
| SU2C3_chr2_ | BIRC9 | V−>I | H−>H | IPPPVCRFMKITIIGRY | IPPPVCRFMKITVIGR |
| 32692980 | | | | GSTNARAK | YGSTNARAK |
| SU2C3_chr1_ | ASPM | I−>S | H−>PU | DMSNTIPDEKVVSTYLS | DMSNTIPDEKVVITYL |
| 457464198 | | | | FLCARLLD | SFLCARLLD |
| SU2C3_chr1_ | NUP137 | C−>Y | PU−>H | INIDEGGWACLVYKEKL | INIDEGGWACLVCKEK |
| 490010827 | | | | HWKIALS | LHWKIALS |
| SU2C3_chr19_ | MEX3D | E−>Q | A−>PU | TVPAAATIWAPFQRAAP | TVPAAATIWAPFERAA |
| 1556093 | | | | LPAFSGCS | PLPAFSGCS |
| SU2C3_chr18_ | SMAD8 | D−>E | A−>A | IEIHLHRALQLLEEVLH | IEIHLHRALQLLDEVL |
| 48604793 | | | | TMPIADPQ | HTMPIADPQ |
| SU2C3_chr2_ | BIRC10 | V−>I | H−>H | IPPPVCRFMKITIIGRY | IPPPVCRFMKITVIGR |
| 32692981 | | | | GSTNARAK | YGSTNARAK |
| SU2C3_chr1_ | ASPM | I−>S | H−>PU | DMSNTIPDEKVVSTYLS | DMSNTIPDEKVVITYL |
| 522557456 | | | | FLCARLLD | SFLCARLLD |
| SU2C3_chr1_ | NUP138 | C−>Y | PU−>H | INIDEGGWACLVYKEKL | INIDEGGWACLVCKEK |
| 555104085 | | | | HWKIALS | LHWKIALS |
| SU2C3_chr19_ | MEX3D | E−>Q | A−>PU | TVPAAATIWAPFQRAAP | TVPAAATIWAPFERAA |
| 1556094 | | | | LPAFSGCS | PLPAFSGCS |
| SU2C3_chr18_ | SMAD9 | D−>E | A−>A | IEIHLHRALQLLEEVLH | IEIHLHRALQLLDEVL |
| 48604794 | | | | TMPIADPQ | HTMPIADPQ |
| SU2C3_chr2_ | BIRCH | V−>I | H−>H | IPPPVCRFMKITIIGRY | IPPPVCRFMKITVIGR |
| 32692982 | | | | GSTNARAK | YGSTNARAK |
| SU2C3_chr1_ | ASPM | I−>S | H−>PU | DMSNTIPDEKVVSTYLS | DMSNTIPDEKVVITYL |
| 587650714 | | | | FLCARLLD | SFLCARLLD |
| SU2C3_chr1_ | NUP139 | C−>Y | PU−>H | INIDEGGWACLVYKEKL | INIDEGGWACLVCKEK |
| 620197343 | | | | HWKIALS | LHWKIALS |
| SU2C3_chr19_ | MEX3D | E−>Q | A−>PU | TVPAAATIWAPFQRAAP | TVPAAATIWAPFERAA |
| 1556095 | | | | LPAFSGCS | PLPAFSGCS |
| SU2C3_chr18_ | SMAD10 | D−>E | A−>A | IEIHLHRALQLLEEVLH | IEIHLHRALQLLDEVL |
| 48604795 | | | | TMPIADPQ | HTMPIADPQ |
| SU2C3_chr2_ | BIRC12 | V−>I | H−>H | IPPPVCRFMKITIIGRY | IPPPVCRFMKITVIGR |
| 32692983 | | | | GSTNARAK | YGSTNARAK |
| SU2C3_chr1_ | ASPM | I−>S | H−>PU | DMSNTIPDEKVVSTYLS | DMSNTIPDEKVVITYL |
| 652743972 | | | | FLCARLLD | SFLCARLLD |
| SU2C3_chr1_ | NUP140 | C−>Y | PU−>H | INIDEGGWACLVYKEKL | INIDEGGWACLVCKEK |
| 685290601 | | | | HWKIALS | LHWKIALS |
| SU2C3_chr19_ | MEX3D | E−>Q | A−>PU | TVPAAATIWAPFQRAAP | TVPAAATIWAPFERAA |
| 1556096 | | | | LPAFSGCS | PLPAFSGCS |
| SU2C3_chr18_ | SMAD11 | D−>E | A−>A | IEIHLHRALQLLEEVLH | IEIHLHRALQLLDEVL |
| 48604796 | | | | TMPIADPQ | HTMPIADPQ |
| SU2C3_chr2_ | BIRC13 | V−>I | H−>H | IPPPVCRFMKITIIGRY | IPPPVCRFMKITVIGR |
| 32692984 | | | | GSTNARAK | YGSTNARAK |

The T cell receptors disclosed herein that are specific for one or more neoantigens can be encoded for and expressed on a T cell. Thus, in one aspect, disclosed herein are T cells (such as, for example, a tumor infiltrating lymphocyte (TIL), chimeric antigen receptor (CAR) T cell, or marrow infiltrating lymphocyte (MIL)) comprising any TCR disclosed herein. The T cells encoding the TCRs disclosed herein can be engineered T cells or T cells from an autologous or allogeneic donor. In one aspect, the T cells are αβ T cells.

It is understood and herein contemplated that T cells (such as, for example, CAR T cells, MILs, and/or TILs) that are reactive to the neoantigens disclosed herein can be administered to a subject with a cancer as a treatment for said cancer. Thus, once identified, the neoantigens can be used to screen for T cells comprising TCRs reactive to the neoantigen and once identified, said T cells can be expanded (in the presence of the neoantigens) and administered to a patient with a cancer. Accordingly, in one aspect, disclosed herein are methods of screening for T cells reactive to a neoantigen, the method comprising: a) obtaining a cancerous tissue sample (such as a tissue sample from a metastasis) from a subject with a cancer; b) fragmenting a first portion of the tissue sample and culturing said first portion; c) expanding tumor infiltrating lymphocytes (TILs) in the cultured first portion. In some aspects, the method can further comprise d) co-culturing the putative neoantigens with the expanded TILs; and e) assaying the co-cultured TILs for reactivity to cancer cells from the subject (for example, assaying for reactivity wherein the reactivity is determined by ELISA, ELISpot, and/or TCRVβ sequencing); wherein reactive TILs indicate are specific to the neoantigen. Alternatively, the method of screening for T cells reactive to neoantigenscan comprise a) obtaining a cancerous tissue sample from a subject with a cancer; b) obtaining a peripheral blood mononuclear cells (PBMCs) from the subject with the cancer; c) isolating T cells from the PBMC from the subject (isolating T cells from the PBMC using any technique known in the art including, but not limited to magnetic cell sorting MACS or FACS); d) co-culturing the putative neoantigens with isolated T cells; and e) assaying the co-cultured isolated T cells for reactivity to cancer cells from the subject (for example, assaying for reactivity wherein the reactivity is determined by ELISA, ELISpot, and/or TCRVβ sequencing); wherein reactive T cells indicate that the putative neoantigen co-cultured with the T cells is a neoantigen.

Because the T cells (such as αβ T cells), TILs, MILs, and/or CAR T cells that bind the neotantigen, the neoantigen can be used to enrich neoantigen specific T cells (such as αβ T cells), TILs, MILs, and/or CAR T cells from a mixed population of T cells. This can be done by co-culturing the neoantigen with the T cells (such as αβ T cells), TILs, MILs, and/or CAR T cells as the T cells (such as αβ T cells), TILs, MILs, and/or CAR T cells specific for the neoantigen will expand and thus comprise a larger portion of the mixed T cell population following culture. Alternatively, the neoantigens can be made to adhere to device or conjugated to a magnetic bead, column, or antibody and neoantigen specific T cells (such as αβ T cells), TILs, MILs, and/or CAR T cells can be bound to the beads, columns, or antibodies and thus separated by non-specific T cells.

In one aspect, it is understood that neoantigens made be administered directly t a subject with a cancer to generate neoantigen reactive T cells, TILs, or MILs in vivo. However, as noted above, the T cells, TILs, MILs, and CAR T cells (such as αβ T cells, TILs, MILS, and CAR T cells comprising TCR reactive to the neoantigens) disclosed herein can be effective in the treatment, inhibition, reduction, amelioration and/or prevention of a cancer. Thus, disclosed herein are methods of treating, inhibiting, reducing, decreasing, ameliorating, and/or preventing a cancer and/or metastasis (such as, for example, a tyrosine kinase inhibitor resistant or EGFR mutated cancer) in a subject comprising administering to the subject any TCRs, T cells, CAR T cells, TILs, and/or MILs disclosed herein that is reactive to one or more neoantigens. For example disclosed herein are methods of treating, inhibiting, reducing, decreasing, ameliorating, and/or preventing a cancer and/or metastasis (such as, for example, a tyrosine kinase inhibitor resistant or EGFR mutated cancer) in a subject comprising administering to the subject a T cell, CAR T cell, TIL, and/or MIL comprising a TCR that recognizes a neoantigen including, but not limited to, neoantigens comprising the sequence DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), YLSFIKILLK (SEQ ID NO: 38), ASPM with a 11250S substitution, NUP133 with a C119Y substitution, ABCC2 with a G664D substitution, ABCC3 with a Y1298C substitution, ABCA12 with a R898I substitution, GPD2 with a D423Y substitution, TP53 with a T155N substitution, MEX3D with a E477Q substitution, SMAD4 with a D537E substitution, TUBB with a T238I substitution, BIRC6 with a V1860I substitution, STIP with a S45F substitution, MAGE-A1, MAGE-A4, MAGE-A5, MAGE-A6, TMPRSS11F with a S306L substitution, and/or any of the neoantigens recited in Table 1. In one aspect, disclosed herein are methods of treating, inhibiting, reducing, decreasing, ameliorating, and/or preventing a cancer and/or metastasis (such as, for example, a tyrosine kinase inhibitor resistant or EGFR mutated cancer) in a subject a TCR or T cell encoding a TCR that comprises the sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), CASSDTARGRNYGYTF (SEQ ID NO: 39), CASSVVGGELFF (SEQ ID NO: 40), CASSPYGTESGANVLTF (SEQ ID NO: 41), CASSQDFRDRAGELFF (SEQ ID NO: 42), CASSQDPSGSYEQYF (SEQ ID NO: 43), CASSQEVGGYTF (SEQ ID NO: 44), CASSQVPGSYEQYF (SEQ ID NO: 45), CSATGTKTNYGYTF (SEQ ID NO: 46), CASSFPAYNEQFF (SEQ ID NO: 47), CSVARVQGASGEQYF (SEQ ID NO: 48), CAWVPGTSGRLVF (SEQ ID NO: 49), CASSLGGPSSPLHF (SEQ ID NO: 50), CASSQLDTYNSPLHF (SEQ ID NO: 51), CAWSEGQSSGNTIYF (SEQ ID NO: 52), CASSSQGRAEAFF (SEQ ID NO: 53), CASSLESLNTEAFF (SEQ ID NO: 54), CASSPQRDGYTF (SEQ ID NO: 55), CASSLGGAGTYEQYF (SEQ ID NO: 56), CASSMDRLYSEAFF (SEQ ID NO: 57), CSAMPVNTGELFF (SEQ ID NO: 58), CASSPVLGQVIYGYTF (SEQ ID NO: 59), CASSIGQNYGYTF (SEQ ID NO: 60), CASKGYRERSYNEQFF (SEQ ID NO: 61), CASSYLVGNTEAFF (SEQ ID NO: 62), CSSVKPQGIGTEAFF (SEQ ID NO: 63), CASSPWATSGRTDTQYF (SEQ ID NO: 64), CASSYVGVQPQHF (SEQ ID NO: 65), CASDGGVSYEQYF (SEQ ID NO: 66), CASSFDASRNEQFF (SEQ ID NO: 67), CASSPHDAGADTEAFF (SEQ ID NO: 68), CASSSFWGYNEQFF (SEQ ID NO: 69), CATSDTRAQGYTF (SEQ ID NO: 70), CATSRDLAGAASNQPQHF (SEQ ID NO: 71), CASSLGLAGVLGETQYF (SEQ ID NO: 72), CASSEYLAGVTEQFF (SEQ ID NO: 73), CSASPVLSYEQYF (SEQ ID NO: 74), CASSLSRMPTNYGYTF (SEQ ID NO: 75), CASSPPAGGLTDTQYF (SEQ ID NO: 76), CASSPGTSPYNEQFF (SEQ ID NO: 77), CASSPDRGSSGNTIYF (SEQ ID NO: 78), CASSYGLNYGYTF (SEQ ID NO: 79), CSAKLAPGGELFF (SEQ ID NO: 70), CSARDNRAGGFAEAFF (SEQ ID NO: 81), CASGGAHNSPLHF (SEQ ID NO: 82), CASSGTGYGGPTGELFF (SEQ ID NO: 83), CASRADRGRNTIYF (SEQ ID NO: 84), CASRKRTGSTDTQYF (SEQ ID NO: 85), CASTHWVGNTEAFF (SEQ ID NO: 86), CASSFTRHRGNEKLFF (SEQ ID NO: 87), CASSHNREGYSNTEAFF (SEQ ID NO: 88), CAISWTSGRALINEQYF (SEQ ID NO: 89), CASSLLLRPNTEAFF (SEQ ID NO: 90), CASRGGTENQPQHF (SEQ ID NO: 91), CSVRTGEGQPQHF (SEQ ID NO: 92), CAWWDAYNSPLHF (SEQ ID NO: 93), CASSKWGANYGYTF (SEQ ID NO: 94), CASTTTPNGQGADTQYF (SEQ ID NO: 95), or CAIRITTGDYGYTF (SEQ ID NO: 96).

It is understood and herein contemplated that the disclosed cancer treatment, inhibition, reduction, amelioration and/or prevention methods can use T cells obtained directly from a subject receiving T cell immunotherapy (i.e., an autologous donor) or from another source (for example an allogeneic donor or engineered T cell). In one aspect, the methods can further comprise obtaining said T cells. Thus, also disclosed herein are methods of treating, inhibiting, reducing, decreasing, ameliorating, and/or preventing a cancer and/or metastasis further comprising obtaining peripheral blood mononuclear cells (PBMCs) from the subject with the cancer or allogeneic donor. T cells can be isolated from the PBMC from the subject using cell sorting techniques known in the art, including but not limited to magnetic cell sorting (MACS) or fluorescence acquired cell sorting (FACS).

In one aspect, disclosed herein are methods of treating, inhibiting, reducing, decreasing, ameliorating, and/or preventing a cancer and/or metastasis wherein the isolated T cells are co-cultured with the putative neoantigens of step e and assayed for reactivity to cancer cells from the subject (for example, assaying for reactivity wherein the reactivity is determined by ELISA, ELISpot, and/or TCRVβ sequencing); wherein reactive T cells indicate that the putative neoantigen co-cultured with the T cells is a neoantigen.

Also disclosed herein are methods of treating, inhibiting, reducing, decreasing, ameliorating, and/or preventing a cancer and/or metastasis in a subject of any preceding aspect, wherein the TILs, MILs, T cells, and/or CAR T cells are expanded in vitro in the presence of one or more of the neoantigens prior to administration of the TILs.

In one aspect, the methods of treating, inhibiting, reducing, decreasing, ameliorating, and/or preventing a cancer and/or metastasis can further comprise administering neoantigen reactive T cells (including, but not limited to, TILs, MILs, and CAR T cells) in combination with any of the disclosed neoantigens or any neoantigen for which the T cell receptor of the T cell is specific. It is understood and herein contemplated that the neoantigens and T cells (including, but not limited to, TILs, MILs, and CAR T cells) can be administered in the same formulation, or separately. When administered separately, the T cells (including, but not limited to, TILs, MILs, and CAR T cells) and neoantigen can be administered concurrently or 1, 2, 3, 4, 5 6, 7, 8, 9, 10, 11, 12, 13, 14, 15, 20, 25, 30, 35, 40, 45, 50, 55, 60, 65, 70, 75, 80, 85, 90, 120 min, 3, 4, 5, 6, 7, 8, 9, 10, 11, 12, 13, 14, 15, 16, 17, 18, 19, 20, 21, 22, 23, 24, 28, 30, 36, 42, 48 hours, 3, 4, 5, 6, 7, 8, 9, 10, 11, 12, 13, 14 days apart with either administration preceding the other. In some aspects, the methods can comprise lymphodepletion prior to administration of the disclosed T cells (such as, for example αβ T cells), TILs, MILs, and/or CAR T cells. Additionally, IL-2 can be administered to the subject following infusion of the T cells (such as, for example αβ T cells), TILs, MILs, and/or CAR T cells.

The disclosed methods and any neoantigen disclosed herein can be used to treat, inhibit, reduce, decrease, ameliorate, and/or prevent any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, non-small cell lung cancer (NSCLC), or pancreatic cancer. In particular, it is understood and herein contemplated that the cancer can be a tyrosine kinase inhibitor resistant or EGFR mutated cancer.

The epidermal growth factor receptor family (ERBB family) comprises four tyrosine kinase receptors: HER-1 (EGFR), Human epithelial receptor (HER)-2 (HER-2)/neu (ERBB2), HER-3 (ERBB3), and HER-4 (ERBB4) (. Following ligand-binding, EGFR receptors homo- and hetero-dimerize and promote autophosphorylation of the intracellular tyrosine kinase domain and initiate molecular cascade of events involved in growth, cell proliferation, differentiation, and survival. Small-molecule receptor tyrosine kinase inhibitors (TKIs) bind to the intracellular catalytic domain of the tyrosine kinase and inhibit receptor autophosphorylation and activation of downstream signaling pathways by competing with adenosine triphosphate (ATP). Gefitinib and erlotinib are the most extensively studied reversible EGFR-TKIs in patients with metastatic NSCLC. The majority of unselected NSCLC patients will not respond to treatment with EGFR-TKIs. Patients of Asian ethnicity, females, never-smokers, or those with adenocarcinoma histology, were initially identified as a population with the most substantial clinical benefit from EGFR-TKIs. The marker of sensitivity to EGFR-TKIs was unknown until 2004 when activating mutations in exon 18, 19, and 21 of the EGFR gene were discovered. The majority of mutations are either point mutations leading to amino acid substitutions (exon 18 and 21) or in-frame deletions (exon 19) clustered around the ATP-binding pocket of the intracellular tyrosine kinase domain. A kinetic analysis of the intracellular domains of mutant EGFR has shown that the mutant receptor compared with a wild-type shows reduced affinity for ATP in the presence of EGFR-TKI.

The EML4-ALK fusion gene is a product of inversion within the short arm of chromosome 2, where ALK (anaplastic large-cell lymphoma kinase) joins EML4 (echinoderm microtubule-associated protein-like 4) to form a fusion gene. The product of EML4-ALK fusion is a chimeric protein with constitutive ALK activity and is detected in 3-6% of unselected NSCLC and especially among never-smokers or light ex-smokers who have adenocarcinoma histology. ALK rearrangements are nearly almost mutually exclusive with EGFR or KRAS mutations, although some rare exceptions exist. ALK-positive NSCLC represents a distinct molecular subtype that can be targeted with ALK-specific treatments. Crizotinib is an oral small-molecule TKI that targets ALK, MET, and ROS1 tyrosine kinases. Crizotinib received accelerated US Food and Drug Administration (FDA) approval for treatment of ALK-positive NSCLC based on an objective response rate of 60% and median PFS of 8-10 months in single-arm studies.

Rearranged in transfection (RET) is a receptor tyrosine with known oncogenic properties in thyroid cancer. Activating amino acid substitutions encoded by germline mutations have been described in multiple endocrine neoplasia (MEN) 2A and 2B, sporadic missense mutations in non-MEN related medullary thyroid cancer, and gene fusions discovered in a subset of papillary thyroid cancers. Oncogenic properties in NSCLC were discovered via transcriptome analysis of banked tumor samples and next generation sequencing, revealing oncogenic KIF5B-RET gene fusions that occur through a paracentric inversion on chromosome 10. These results have been replicated with FISH analysis, with overall incidence of approximately 1-2%, with enrichment strategies for lung adenocarcinoma patients negative for other known biomarkers (ALK, EGFR, KRAS, ROS1) yielding rates up to ˜15%. Additional gene fusion partners have been discovered, including CCD6, NCOA4, and TRIM33. There is a higher prevalence of RET+ NSCLC amongst never/light smoker and younger cohorts. The majority of RET+ NSCLC occur in patients with adenocarcinoma and the mutation occurs more frequently in poorly differentiated tumors.

MET is a proto-oncogene that encodes for the heterodimeric transmembrane MET tyrosine receptor kinase. Its only known ligand-hepatocyte growth factor (HGF). Binding of HGF to the MET receptor activates the tyrosine kinase and downstream signaling pathways including PI3K/AKT, Ras-Rac/Rho, mitogen-activated protein kinase (MAPK), and phospholipase C (PLC) involved in cell motility and invasion. The MET receptor is expressed in approximately 40-50% of NSCLC tumors; high levels of receptor expression, as well as high MET gene copy number are independent prognostic factors of poor outcome in patients with resected NSCLC. MET amplification is recognized as one of the potential molecular mechanisms of acquired resistance in EGFR-mutated NSCLC to EGFR-TKIs.

ROS-1 is an orphan receptor tyrosine kinase that is phylogenetically related to ALK. ROS-1 chromosomal rearrangements with CD74, EZR, SLC24A2, and FIG genes define a new genomic driver in 1-2.5% of NSCLC patients. Clinical characteristics of NSCLC patients with ROS-1 rearrangements are similar to patients with ALK-rearranged NSCLC—more commonly seen in patients of Asian ethnicity, young age (median age 49.8 years), female sex, never-smokers, and adenocarcinoma histology. ROS-1 rearrangements appear mutually exclusive of other known oncogenic drivers like EGFR, KRAS, HER-2, ALK, RET, and MET aberrations. Pre-clinical data showed activity of ALK inhibitors (i.e., crizotinib and TAE684) in ROS rearranged NSCLC cell lines given the high degree of homology between ALK and ROS-1 tyrosine kinase domains. This led investigators to assess the benefit of crizotinib in this unique patient subset. Efficacy has been demonstrated with an overall response rate of 56% and 6-month PFS of 71% in 25 evaluable patients. There are a number of currently ongoing phase I and II studies investigating activity of crizotinib, dual ALK/ROS1 inhibitor PF-06463922, and ceritinib in ROS-1-rearranged NSCLC.

The RAS oncogene family, HRAS, KRAS, and NRAS, encodes intracellular transducer proteins (small GTPases) that are involved in transmitting signals from extracellular growth factor receptors like EGFR to the cell. As G proteins, they are located on the intracellular side of the plasma membrane, bind guanine nucleotides, and have GTP-ase activity. In the resting state, RAS proteins are bound to GDP and are inactive. Upon exchange of GDP to GTP, the RAS-GTP complex activates multiple downstream pathways (MAPK, STAT, and PI3K) that regulate cell proliferation, motility, and apoptosis. After a short period, the signaling configuration of RAS is halted by intrinsic GTP-ase activity. Activating RAS mutations prevent GTP hydrolysis to GDP, thus the RAS protein is rendered constitutively active with uncontrolled activation of downstream signaling pathways.

The KRAS gene is one of the first described oncogenes. KRAS mutations are present in approximately 30% of lung adenocarcinomas and less commonly in squamous NSCLC (˜5%). They are found more frequently in Caucasians with lung cancer than in the Asian population and in current- or ex-smokers when compared with never-smokers. Most KRAS mutations in NSCLC are single amino acid substitutions in codon 12 (80%) and to a lesser extent in codons 13 and 61. In current- or ex-smokers, KRAS mutations are usually transversions (pyrimidine nucleotide is exchanged for purine or vice versa; e.g., G →T or G →C) and transitions in never-smokers (purine nucleotide is exchanged for another purine or pyrimidine for another pyrimidine; e.g., G →A or C →T). KRAS mutations are nearly always mutually exclusive with EGFR and BRAF mutations although rare co-existence of EGFR and KRAS mutations has been observed. KRAS mutations co-exist with PIK3CA mutations in approximately 19% of PIK3CA-mutant NSCLC.

It has been postulated for over 20 years that KRAS-mutant NSCLC may be associated with poor outcome. However, multiple studies have shown conflicting results due to heterogeneity among the studies, including tumor type, stage, treatment, and study end points. A meta-analysis of 28 studies published in 2005 demonstrated that KRAS mutation was a significant prognostic marker when polymerase chain reaction sequencing was used as a detection method. Recently published results of a LACE-Bio pooled retrospective analysis reported no prognostic or predictive (in regard to benefit from adjuvant chemotherapy) effect of KRAS mutations in patients with resected NSCLC. A subset analysis of patients with NSCLC with KRAS codon 13 mutations suggests that adjuvant chemotherapy may have a deleterious effect in this subgroup, but needs to be further validated (HR—5.78; 95% CI, 2.06-16.2). In the absence of prospective, large, randomized clinical trials, KRAS mutation status in NSCLC cannot be used as a prognostic nor predictive biomarker for treatment with exception of negative predictive value of KRAS mutations and response to EGFR-TKI.

Direct inhibition of KRAS has been unsuccessful so far due to its molecular and functional complexity. The activation of the RAS-RAF-MEK-ERK signaling pathway as a consequence of KRAS mutations renders it an attractive target for small-molecule inhibition in KRAS-mutated NSCLC. Given the critical location in this signaling pathway, MEK has been recognized as an important target, downstream from KRAS, for anti-cancer therapy.

KRAS mutations in NSCLC, despite being the most common, remain the most intriguing and elusive of therapeutic targets. At present, targeted treatment is not available for KRAS-mutated NSCLC outside clinical trials. However, novel agents targeting downstream effector signaling pathways are under clinical development.

Accordingly, in one aspect disclosed herein are methods of treating, inhibiting, reducing, decreasing, ameliorating, and/or preventing a cancer and/or metastasis in a subject, wherein the cancer comprises a mutation, overexpression, activation, inactivation, or fusion of an epithelial growth factor receptor (EGFR) family gene (such as, for example, Human epithelial receptor (HER)-2 (HER-2), HER-3, or HER-4), an anaplastic lymphoma kinase (ALK) gene (such as, for example, a fusion of ALK and echinoderm microtubule-associated protein-like 4 (EML4), kinesis family member 5B (KIFSB), 5-aminoimidazole-4-carboxamide ribonucleotide transformylase/inosine 5′-monophosphate cyclohydrolase (ATIC), carbamoyl-phosphate synthetase 2, aspartate transcarbamylase, and dihydmorotase (CAD), Clathrin heavy chain 1 (CLTC), Dynactin subunit 1 (DCTN1), Fibronectin (FM), Huntingtin-interacting protein 1 (HIP1), Kinesin light chain 1 (KLC1), Nucleophosmin (NPM1), Tropomyosin alpha-3 (TPM3), Tropomyosin alpha-4 (TPM4), TFG, Striatin (STRN), Sequestosome-1 (SQSTM1), or RAs-related Nuclear (RAN) binding protein 2 (RANBP2)), a c-ROS oncogene 1 (ROS-1) gene (such as, for example, a fusion of ROS1 and SLC34A2, CD74, SDC4, TPM3, EZR, LRIG3, KDLER2, CCDC6, YWHAE, TFG, or CEP85L), a MET, a Fibroblast growth factor receptor 1 (FGFR1), B Rapidly Accelerated Fibrosarcoma (BRAF) gene, neurotrophic receptor tyrosing kinase (NTRK) gene, a Rearranged in Transfection (RET) gene (such as, for example, a fusion of RET and kinesis family member 5B (KIF5B), Coiled-coil domain-containing protein 6 (CCDC6), NCOA, EPHA5, PICALM, TRIM33, CUX1, or KIAA1468), or a Kirsten rat sarcoma viral oncogene homolog (KRAS).

In one aspect, it is understood the treatment of cancer does not need to be limited to the administration of neoantigens and/or neoantigen-specific T cells, but can include the further administration of anti-cancer agents to treat, inhibit, reduce, decrease, ameliorate, and/or prevent a cancer and/or metastasis. Anti-cancer therapeutic agents (such as chemotherapeutics, immunotoxins, peptides, and antibodies) that can be used in the methods of treating, inhibiting, reducing, decreasing, ameliorating, and/or preventing a cancer and/or metastasis and in combination with any of the disclosed neoantigens or any CAR T cells, TIL, or MIL specific for said neoantigen can comprise any anti-cancer therapeutic agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

In one aspect, it is understood that the additional anti-cancer agent administered in addition to the any TCRs, T cells, CAR T cells, TILs, and/or MILs disclosed herein for the treatment, decrease, reduction, inhibition, amelioration, and/or prevention of a cancer or metastasis can be an immune checkpoint inhibitor. Thus, in one aspect, disclosed herein are methods of treating, inhibiting, reducing, decreasing, ameliorating, and/or preventing a cancer and/or metastasis in a subject further comprising administering to the subject an immune checkpoint inhibitor, wherein the checkpoint inhibitor comprises a PD1/PDL1 blockade inhibitors and/or CTLA4/B7-1 or 2 inhibitors (such as, for example, PD-1 inhibitors lambrolizumab, OPDIVO® (Nivolumab), KEYTRUDA® (pembrolizumab), and pidilizumab; PD-L1 inhibitors BMS-936559, TECENTRIQ® (Atezolizumab), IMFINZI® (Durvalumab), and BAVENCIO® (Avelumab); and CTLA-4 inhibitors YERVOY (ipilimumab).

As noted above, the cancers to be treated, inhibited, reduced, decreased, ameliorated, and/or prevented by the methods disclosed herein can comprise a tyrosine kinase inhibitor resistant cancer. It is understood and herein contemplated that administration of the disclosed TCRs, T cells, CAR T cells, TILs, and/or MILs can make a cancer that is resistant to a tyrosine kinase inhibitor more susceptible to said treatment. Thus, in one aspect, disclosed herein are methods of treating, inhibiting, reducing, decreasing, ameliorating, and/or preventing a cancer and/or metastasis in a subject further comprising administering to the subject crizotinib, ceritinib, alectinib, brigatinib, vemurafenib, dabrafenib, afatinib, Tivantinib, AMG 102, ficlatuzumab, cabozantinib, foretinib, ponatinib, onartuzumab, LKD378, AP26113, TSR-011, Selumetinib, TAE684, Trametinib, barbozatinib, gefitinib, erlotinib, paptinib, vandetanib, afatinib, osimertinib, lenvatinib, nintedanib, pazopanib, regorafenib, sorafenib, sunitinib, bosutinib, dasatinib, imatinib, nilotinib, and/or ponatinib.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

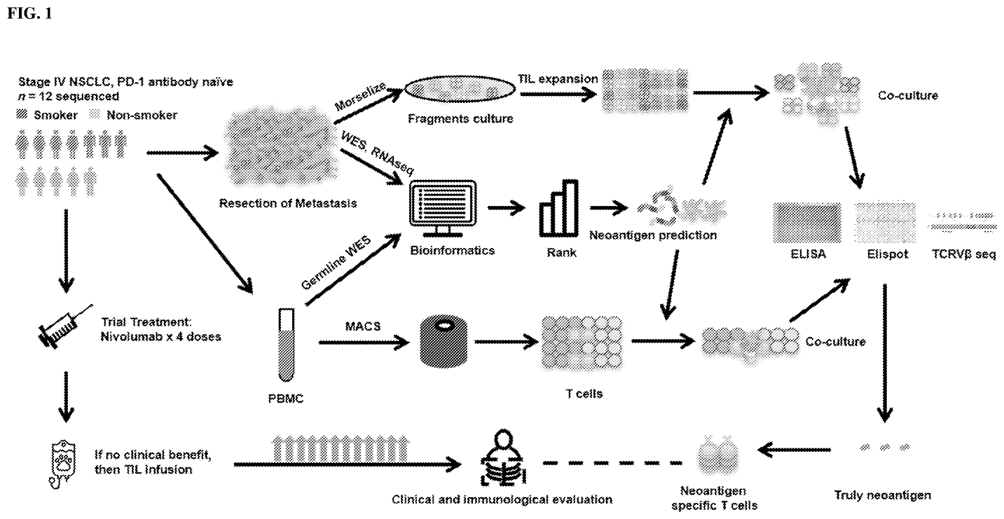

As shown in FIG. 1, this process is developed to identify neoantigens and hen utilize neoantigens to isolate neoantigen specific TILs for the treatment of a cancer. PBMCs and/or tissue resections were collected from patients before and after combination therapy. PBMC were subjected to magnetic cell separation (MACS) to isolate T cells from the PBMC. Concurrently, metastatic tissue resections were either fragmented or subjected to sequence analysis through whole exosome sequencing (WES) or RNAseq analysis. The fragmented sections were cultured to expand tumor infiltrating lymphocytes. Bioinformatic analysis was performed on the sequencing data for the determination of the number and sequence of each of the productive unique VP gene identified within each sample and the degree of clone sharing between samples.

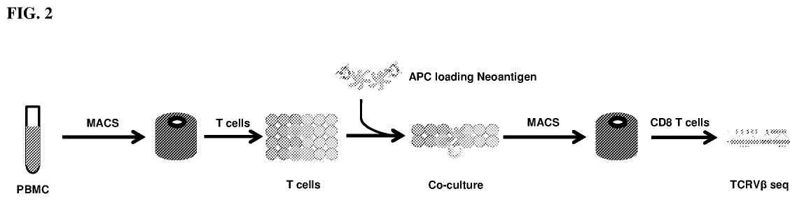

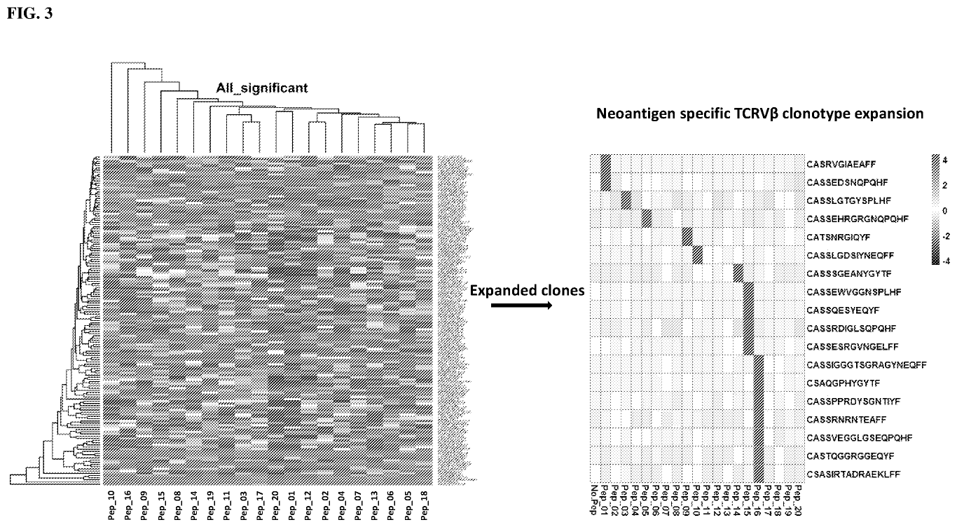

To assay the reactivity of the unique peptides TCRVβ sequencing was used. Briefly, potential neoantigens were contacted with TILs isolated from either expanded from fragmented tissue resections or isolated via MACS from PBMC and the TVRβ sequencing performed to show reactivity. (See FIGS. 2 and 3).

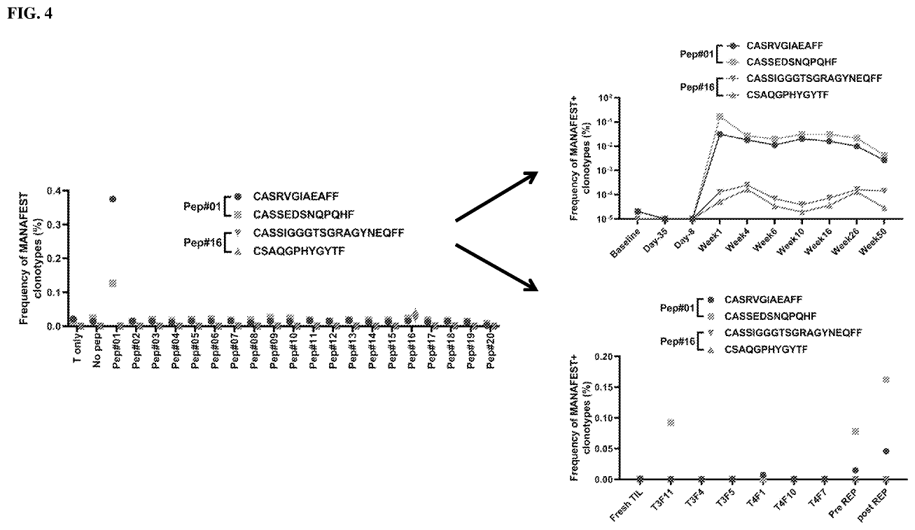

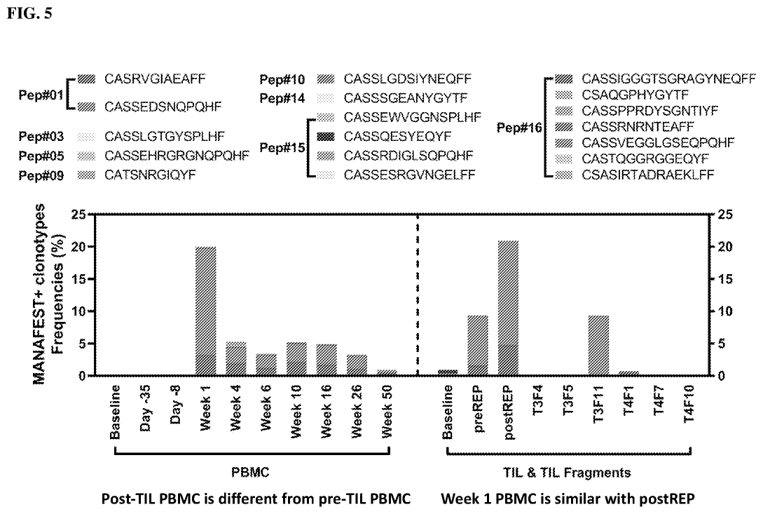

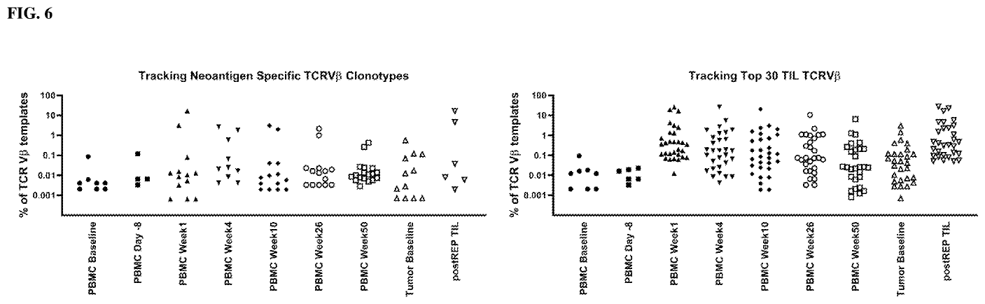

To define the immunogenicity of every individual candidate predicted in the analysis of mutational burden analysis, the presence of reactive T cell clones in peripheral blood were individually quantified in MANAFEST analysis. MANAFEST is a novel, scalable method to evaluate candidate tumor neoantigens for their ability to induce T cell responses (more sensitive and specific than conventional ELISPOT or ELISA). Briefly, up to 30 mutations associated neoantigen candidates per tumor (MANAs) were synthesized (New England Peptide, Inc) for reactivity analysis. The T cell fraction of PBMCs collected before and 3 and 7 weeks after initial treatment for every patient were separated using beads. The non-T cell fraction is then gamma-irradiated and autologous T and irradiated non-T cells are put back together. Predicted neoantigens are then added to specific wells in triplicate, along with cytokines (such as, IL-2, IL-7 and IL-15). On days 3 and 7, half the medium is replaced with fresh medium containing cytokines. CD8 T cells are then separated using a CD4 positive selection kit and subjected to ImmunoSeq analysis. Controls include CEF peptides (epitopes from common viral infections) or no peptide. TCR beta chains expanded >10-fold in response to individual peptides (but not in response to other neoantigens or control CEF peptides) are considered positive. (See FIGS. 4 and 5). FIG. 4 shows MANAFEST+data for various clonotypes. The data reveal that Neoantigen specific clones exist in some of TIL fragments as well as postREP. Additionally, the frequencies of neoantigens increased dramatically after TIL infusion and were very low in baseline tumors (FIG. 6).

Following the experiments provided herein certain conclusions could be made. First, from a detection on procedural basis, peptide-based antigen screening can effectively detect viral peptide controls. Additionally, irradiated non-T cells are preferred as APCs and the supernatant IFN-γ levels are best detected on Day 3 with less background IFN-γ. Also, peptide screening can effectively identify private tumor neoantigens using both PBMCs and TIL. Interestingly, TILs have a higher sensitivity than PBMCs for tumor neoantigen detection and cultured lung cancer TILs were shown to retain autologous tumor recognition.

The data provided herein also show that TIL infusion helps increase neoantigen-specific TCR clonotypes. Additionally, TIL abundant TCR clonotypes, including neoantigen specific ones, can be retained in recipient blood (recurrent tumor to be determined) for a long time. As much of this data was done in patients with non-small cell lung carcinoma, the current neoantigen screening approach is feasible in lung cancer patients.

To determine the sequence and persistence of infused TRVβ clonotypes serial variable chain sequencing was performed (see Table 2). The list in Table 2 shows the T cell receptor beta variable chains that were discovered, which recognize specific tumor mutations. These T cell receptors are derived from two lung cancer patients who had durable remission following infusion of tumor-infiltrating T cells that targeted multiple tumor antigens, including the mutations listed. The TCR sequences were derived by peptide co-culture experiments. In this experiment, peptides corresponding to the mutation of interest of incubated with autologous human dendritic cells, autologous human T cells, IL-7, IL-15, and IL-2, and media. T cell receptor sequencing TCR-VB (hsTCRB kit) is performed after 10 to 14 days of co-culture. CDR3 sequences are compared to multiple no-peptide controls to assess whether any have expanded, and selected based on frequency in peripheral blood. The T cells used can be either tumor-infiltrating lymphocytes (TIL) or peripheral blood T cells.

FIG. 7 shows the high degree of overlap between the infused TIL, and circulating clonotypes at post-infusion timepoints. This indicates that TIL persisted in the peripheral blood. Shown are two representative patients. Note that Patient 3 had progressive disease noted at Day +350, which was resected. TIL cultured from the tumor had high overlap. Looking at the dynamics of the response (FIG. 8), note that there is an initial expansion of the neoantigen specific T cell clonotypes relative to other T cells after infusion. Taken altogether, the antigen-specific T cells are 21% of the total TIL infused.

TABLE 2
TCR CDR3 clonotypes matched to tumor mutations

| | | | | | | | | | Petide | | |
|---|---|---|---|---|---|---|---|---|---|---|---|
| | | | | | | | | | (# | Fold- | |
| | | Mu- | | | | | | | depends | expansion | |
| Patient | | tation | | RNA | DNA | TCR-VB VDJ | T | Pep- | onLong | vs. no- | Experiment |
| Trial | | Amino | Mutation | VAF in | VAF in | CDR3 Amino | cell | tide | or | peptide | T cell |
| ID | Gene | Acid | Position | Tumor | Tumor | Acid Sequence | Type | Type | Short) | (average) | source |
| 3 | NUP133 | C119Y | p.C119Y | 29.17% | 14.29% | CASRVGIAEAFF | CD8 | Short | 1S | 24.16 | Peripheral |
| | | | (NM_018230) | | | (SEQ ID NO: 39) | | | | | Blood |
| 3 | NUP133 | C119Y | p.C119Y | 29.17% | 14.29% | CASSEDSNQPQHF | CD8 | Short | 1S | 4.70 | Peripheral |
| | | | (NM_018230) | | | (SEQ ID NO: 40) | | | | | Blood |
| 3 | NUP133 | C119Y | p.C119Y | 29.17% | 14.29% | CASSPYGTESGANVLTF | CD8 | Short | 1S | 4.16 | Peripheral |
| | | | (NM_018230) | | | (SEQ ID NO: 41) | | | | | Blood |
| 3 | SMAD4 | D537E | p.D537E | 58.33% | 19.53% | CASSQDFRDRAGELFF | CD8 | Long | 14L | 9.80 | Peripheral |
| | | | (NM_005359) | | | (SEQ ID NO: 42) | | | | | Blood |
| 3 | SMAD4 | D537E | p.D537E | 58.33% | 19.53% | CASSQDPSGSYEQYF | CD8 | Long | 14L | 5.95 | Peripheral |
| | | | (NM_005359) | | | (SEQ ID NO: 43) | | | | | Blood |
| 3 | NUP133 | C119Y | p.C119Y | 29.17% | 14.29% | CASSQEVGGYTF | CD4 | Long | 2L | 9.37 | Peripheral |
| | | | (NM_018230) | | | (SEQ ID NO: 44) | | | | | Blood |
| 3 | MEX3D | E447Q | p.E477Q | 59.38% | 25.23% | CASSQVPGSYEQYF | CD4 | Long | 10L | 44.08 | Peripheral |
| | | | (NM_001174118) | | | (SEQ ID NO: 45) | | | | | Blood |
| 3 | TUBB | T238I | p.T238I | 23.06% | 14.89% | CSATGTKTNYGYTF | CD4 | Long | 8L | 8.46 | TIL |
| | | | (NM_178014) | | | (SEQ ID NO: 46) | | | | | |
| 3 | TUBB | T238I | p.T238I | 23.06% | 14.89% | CASSFPAYNEQFF | CD4 | Long | 8L | 8.35 | Peripheral |
| | | | (NM_178014) | | | (SEQ ID NO: 47) | | | | | Blood |
| 3 | TUBB | T238I | p.T238I | 23.06% | 14.89% | CSVARVQGASGEQYF | CD4 | Long | 8L | 14.66 | TIL |
| | | | (NM_178014) | | | (SEQ ID NO: 48) | | | | | |
| 3 | TUBB | T238I | p.T238I | 23.06% | 14.89% | CAWVPGTSGRLVF | CD4 | Long | 8L | 16.10 | Peripheral |
| | | | (NM_178014) | | | (SEQ ID NO: 49) | | | | | Blood |
| 3 | ASPM | H250S | p.H250S | 35.71% | 13.11% | CASSLGGPSSPLHF | CD4 | Long | 1L | 6.10 | Peripheral |
| | | | (NM_001206846) | | | (SEQ ID NO: 50) | | | | | Blood |
| 3 | BIRC6 | V1860I | p.V1860I | 17.58% | 18.60% | CASSQLDTYNSPLHF | CD4 | Long | 20L | 8.91 | Both blood |
| | | | (NM_016252) | | | (SEQ ID NO: 51) | | | | | and TIL |
| 3 | STIP | S45F | p.S45F | 28.77% | 13.10% | CAWSEGQSSGNTIYF | CD4 | Long | 7L | 12.96 | Peripheral |
| | | | (NM_006819) | | | (SEQ ID NO: 52) | | | | | Blood |
| 3 | STIP | S45F | p.S45F | 28.77% | 13.10% | CASSSQGRAEAFF | CD4 | Long | 7L | 17.64 | TIL |
| | | | (NM_006819) | | | (SEQ ID NO: 53) | | | | | |
| 3 | STIP | S45F | p.S45F | 28.77% | 13.10% | CASSLESLNTEAFF | CD4 | Long | 7L | 6.38 | TIL |
| | | | (NM_006819) | | | (SEQ ID NO: 54) | | | | | |
| 3 | STIP | S45F | p.S45F | 28.77% | 13.10% | CASSPQRDGYTF | CD4 | Long | 7L | 23.02 | TIL |
| | | | (NM_006819) | | | (SEQ ID NO: 55) | | | | | |
| 3 | STIP | S45F | p.S45F | 28.77% | 13.10% | CASSLGGAGTYEQYF | CD4 | Long | 7L | 31.83 | TIL |
| | | | (NM_006819) | | | (SEQ ID NO: 56) | | | | | |
| 3 | STIP | S45F | p.S45F | 28.77% | 13.10% | CASSMDRLYSEAFF | CD4 | Long | 7L | 94.26 | Both blood |
| | | | (NM_006819) | | | (SEQ ID NO: 57) | | | | | and TIL |
| 9 | GPD2 | D423Y | p.D423Y | 29.03% | 9.33% | CSAMPVNTGELFF | CD8 | Long | 31 | 9.23 | Peripheral |
| | | | (NM_000408) | | | (SEQ ID NO: 58) | | | | | Blood |
| 9 | ABCC3 | Y1298C | p.Y1298C | 38.10% | 20.93% | CASSPVLGQVIYGYTF | CD4 | Long | 23 | 16.00 | Peripheral |
| | | | (NM_003786) | | | (SEQ ID NO: 59) | | | | | Blood |
| 9 | GPD2 | D423Y | p.D423Y | 29.03% | 9.33% | CASSIGQNYGYTF | CD4 | Long | 31 | 29.74 | Peripheral |
| | | | (NM_000408) | | | (SEQ ID NO: 60) | | | | | Blood |
| 9 | GPD2 | D423Y | p.D423Y | 29.03% | 9.33% | CASKGYRERSYNEQFF | CD4 | Long | 31 | 55.78 | Peripheral |
| | | | (NM_000408) | | | (SEQ ID NO: 61) | | | | | Blood |
| 9 | GPD2 | D423Y | p.D423Y | 29.03% | 9.33% | CASSYLVGNTEAFF | CD4 | Long | 31 | 70.97 | Peripheral |
| | | | (NM_000408) | | | (SEQ ID NO: 62) | | | | | Blood |
| 9 | GPD2 | D423Y | p.D423Y | 29.03% | 9.33% | CSSVKPQGIGTEAFF | CD4 | Long | 31 | 18.00 | Peripheral |
| | | | (NM_000408) | | | (SEQ ID NO: 63) | | | | | Blood |
| 9 | GPD2 | D423Y | p.D423Y | 29.03% | 9.33% | CASSPWATSGRTDTQYF | CD4 | Long | 31 | 95.75 | Peripheral |
| | | | (NM_000408) | | | (SEQ ID NO: 64) | | | | | Blood |

### 2. Example 2: Complete Responses Induced by Tumor-Infiltrating Lymphocyte Treatment in Anti-PD-1 Resistant Metastatic Lung Cancer

Adoptive cell therapy using live tumor-infiltrating lymphocytes (TIL) cultured from a patient's tumor is a potent form of immunotherapy. TIL treatment has caused durable complete responses in a subset of patients with metastatic melanoma. Like melanoma, non-small cell lung cancers (NSCLC) also contain T cells that recognize tumor antigens. Although restricted in a suppressed state, these T cells can be reactivated to destroy tumor cells by therapeutic blockade of immune checkpoint proteins, such as programmed death receptor-1 (PD-1). However, anti-PD1 clinical responses only occur in a minority of NSCLC patients, and eventual progression is common. Therefore, more effective combination immunotherapy is needed for metastatic NSCLC.

Ex vivo expansion of TIL can release T cells from a suppressive microenvironment and re-activate them to target the tumor. By this method, billions of activated T cells can be produced and infused back into a patient. In contrast to recombinant monoclonal technology such as chimeric antigen receptors (CAR-T), TIL is composed of polyclonal cells capable of targeting multiple tumor antigens. Since TIL are derived from native genetically unmodified cells, complications due to engagement of normal host cells is uncommon. Durable regressions with TIL have previously been reported in a variety of epithelial malignancies, including cholangiocarcinoma, cervical, colorectal, and breast cancer. We hypothesized that TIL would be feasible and have clinical activity in metastatic NSCLC.

We performed a phase I pilot trial of nivolumab followed by TIL in metastatic NSCLC patients, with the primary endpoint of safety, and secondary endpoints of response rate and survival. We harvested tumors from patients naïve to PD(L)1 blockade, to reduce the proportion of terminally differentiated T cells in culture. Those with tumor enlargement or progression proceeded to receive TIL. Infusion of TIL in combination with lymphodepletion and interleukin (IL-2) mediated tumor regressions in several patients, including complete responses.

a) Results

(1) Patient Characteristics

Twenty patients were enrolled. Among them, 50% were current or former smokers. The median age was 54 years (range 38-75) and median PD-L1 proportion score was 6% (Table 3). Median estimated tumor mutation burden (TMB) was 4.5 mutations per megabase of DNA (range 0.8-25). The predominant enrolled histology was lung adenocarcinoma. Most patients had bulky disease, with a mean sum of target lesion diameters of 8.5 cm prior to TIL treatment.

TABLE 3
Baseline characteristics of all enrolled patients.

| | | | | PD-L1 | TMB | Smoking | Driver | Harvested | Prior |
|---|---|---|---|---|---|---|---|---|---|
| ID | Histology | Age | Sex | TPS | mut/MB | pack-year | oncogene(s) | Metastasis | Therapy |
| 01 | Adenocarcinoma | 48 | M | 11% | 0.8 | 0 | CCDC6-RET | Supraclavicular LN | None |
| 02 | Adenocarcinoma | 54 | M | 0% | 9 | 30 | KRAS G12V | Supraclavicular LN | Carboplatin and pemetrexed |
| 03 | Adenocarcinoma | 69 | F | 0% | 1 | 1.2 | EGFR Ex20 ins | Paratracheal LN | RT |
| 04 | Adenosquamous | 44 | F | 85% | 3 | 0 | CD74-ROS1 | Supraclavicular LN | Crizotinib* |
| 05 | Adenocarcinoma | 63 | F | 1% | 4 | 17.5 | KRAS G12C | Pleural nodule | None |
| 07 | Adenocarcinoma | 49 | F | 0% | 4 | 0 | EGFR L861Q | Pleural nodule | None* |
| 08 | Squamous | 49 | M | 0% | 5 | 0 | None | Axillary LN | RT |
| 09 | Adenocarcinoma | 56 | F | 10% | 25 | 30 | None | Supraclavicular LN | None |
| 12 | Adenocarcinoma | 50 | F | 20% | 4 | 0 | EML4-ALK | Pleural nodule | None* |
| 13 | Squamous | 69 | F | 17% | 18 | 40 | None | Lung nodule | None |
| 14 | Adenocarcinoma | 53 | F | 100% | 16 | 34 | None | Supraclavicular LN | Carboplatin and paclitaxel, SBXRT |
| 15 | Adenocarcinoma | 75 | M | 0% | 3 | 25 | KRAS G12C | Pleural nodule | None |
| 16 | Squamous | 66 | M | 0% | 7 | 0 | None | Pleural nodule | Carboplatin and paclitaxel |
| 25 | Adenocarcinoma | 61 | F | 2% | 6 | 0 | EGFR Ex19 del | Pleural nodule | Afatinib, osimertinib |
| 26 | Squamous | 60 | M | 80% | 15 | 40 | PI3CKA | Chest wall met | Cisplatin and VP16, carboplatin |
| 29 | Adenocarcinoma | 45 | M | 90% | 5 | 32 | KRAS G12C | Supraclavicular LN | None |
| 30 | Adenocarcinoma | 38 | M | 100% | 2.5 | 0 | EML4-ALK | Cervical LN | Cisplatin and pemetrexed, alectinib |
| 31 | Adenocarcinoma | 69 | M | 97% | 6 | 40 | KRAS G12V | Pleural nodule | None |
| 32 | Adenocarcinoma | 45 | F | 0% | 3 | 0 | EGFR Ex19 del | Pleural nodule | Osimertinib |
| 33 | Squamous | 42 | M | 0% | 1 | 0 | EWSR1-CREM | Pleural nodule | None |

| | | | | | | | |
|---|---|---|---|---|---|---|---|
| Median: | 54 | 6% | 4.5 | 9 | — | — | 0-1 |

Footnotes:
Abbreviations:
TPS denotes tumor proportion-score by 22C3 PD-L1 score;
LN, lymph node;
TMB; estimated tumor mutation burden;
RT, radiation;
SBXRT, stereotactic body radiation; and
mut/MB, mutations per megabase.
*Indicates patient with initial negative testing for ALK or EGFR by plasma and/or FISH testing, only identified after next-generation sequencing of excised tumor after enrollment

(2) Outcome after TIL Harvest and Initial Nivolumab

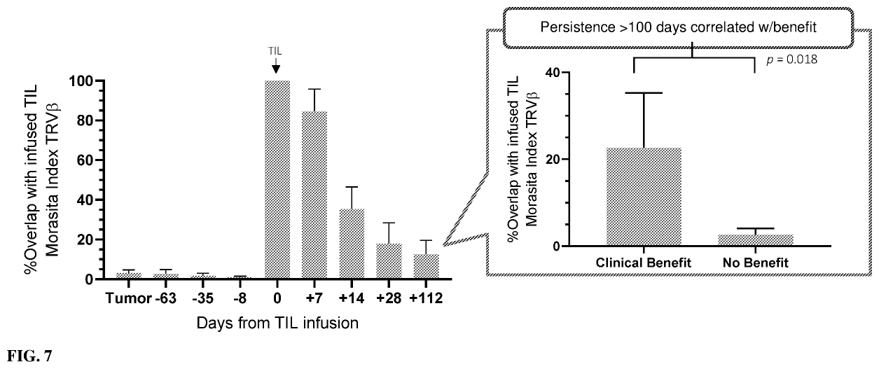

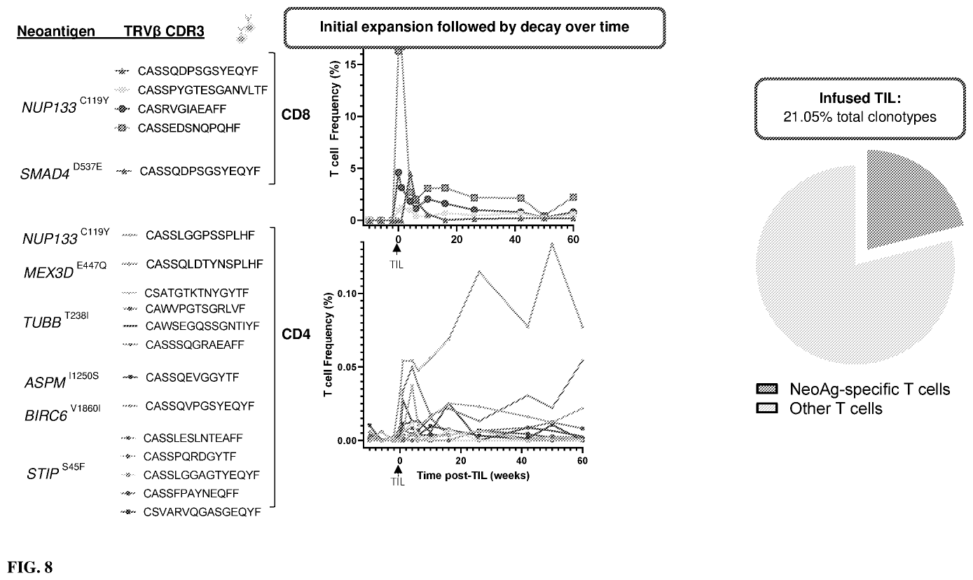

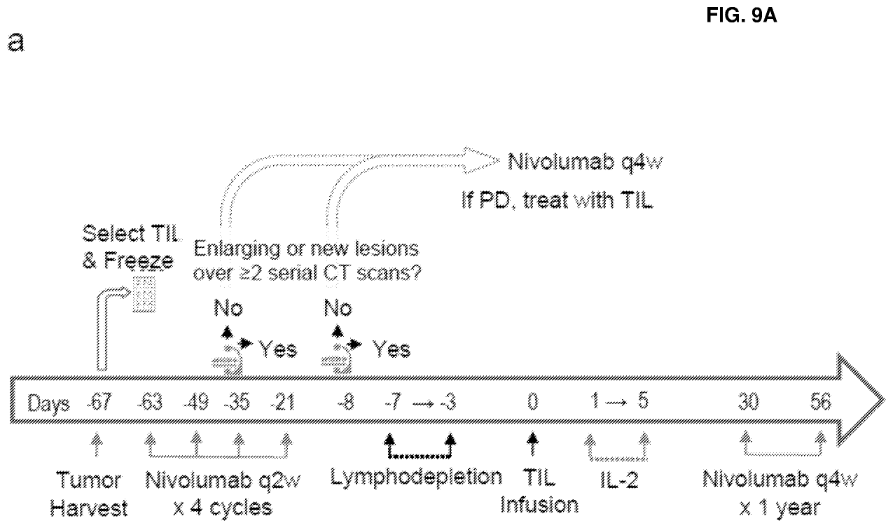

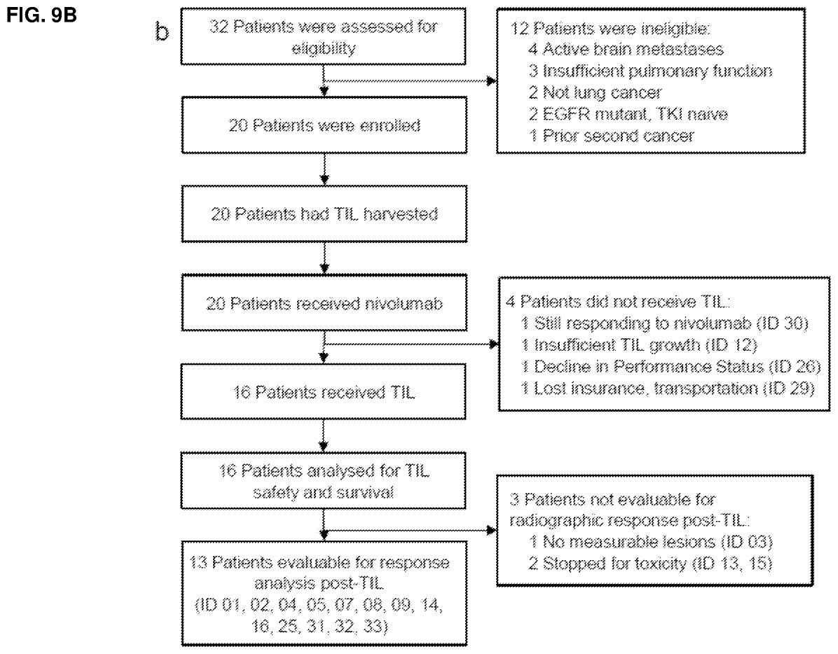

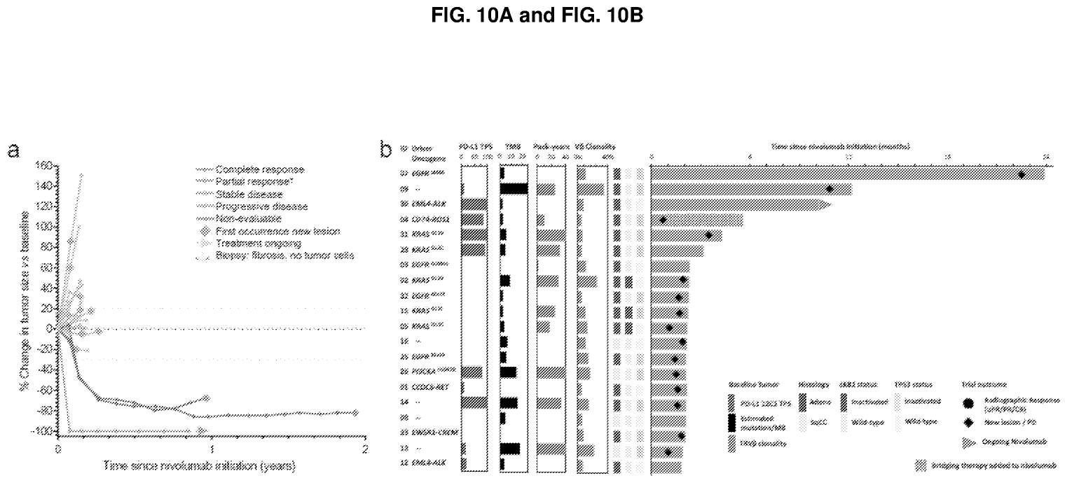

Ninety percent of patients were discharged to home within one day or less after the excisional biopsy for TIL harvest. Nivolumab treatment did not have any previously unreported adverse effects. After excisional biopsy of the metastasis, all patients were treated with at least 4 cycles of intravenous nivolumab at a dose of 240 mg every 2 weeks (FIG. 9a). If patients had evidence of clinical benefit after two sequential CT scans, then nivolumab was continued until progression. If there was evidence of progression, as defined by tumor enlargement or new lesions, then we proceeded to administer lymphodepletion and TIL treatment. At least two sequential CT scans were required to reduce the possibility of confounding error due to pseudoprogression. The majority of patients had tumor enlargement or new lesions (FIG. 10a). Three patients had eventual PR or CR with initial nivolumab, and therefore nivolumab was continued. Two of these patients with an initial nivolumab benefit eventually had biopsy-proven progression while on nivolumab (FIG. 10b). These two were subsequently treated with TIL.

(3) Feasibility of TIL

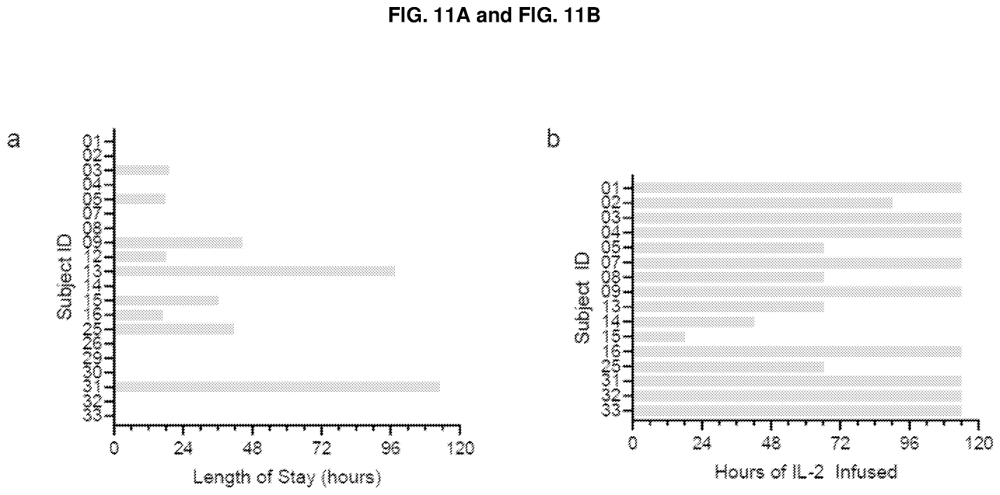

TIL were successfully expanded for 95% of patients to a median dose of 95 billion CD3⁺ cells (range 4.3-175). Of those tested, specific reactivity to autologous tumor was detected in oligoclonal TIL cultures within 14 of 18 patients. Four patients did not receive TIL due to reasons outlined (FIG. 9b). In total, 16 patients received lymphodepletion chemotherapy with cyclophosphamide and fludarabine followed by TIL infusion and IL-2 for 5 days. All of these patients received full course lymphodepletion chemotherapy without dose modification. All treated patients initiated infusional IL-2, and the majority (56%) received all planned doses (FIG. 11b).

(4) Clinical Activity

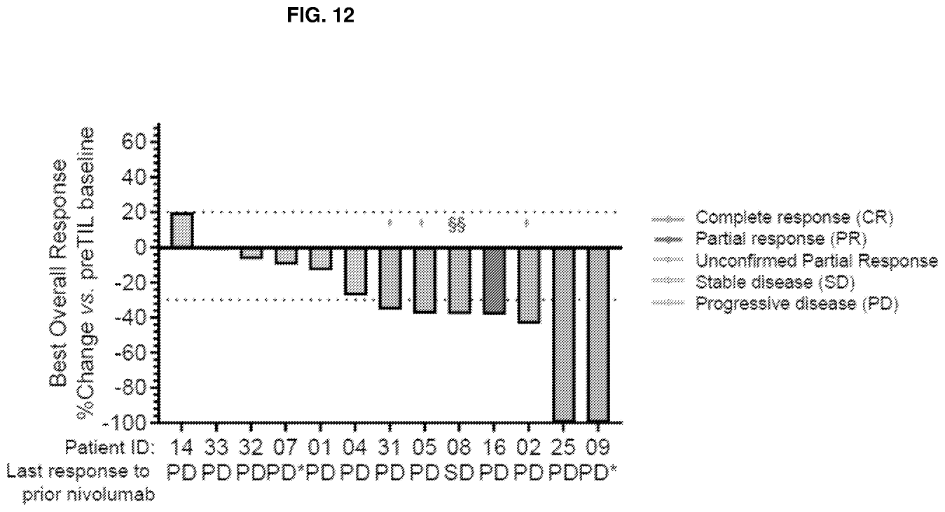

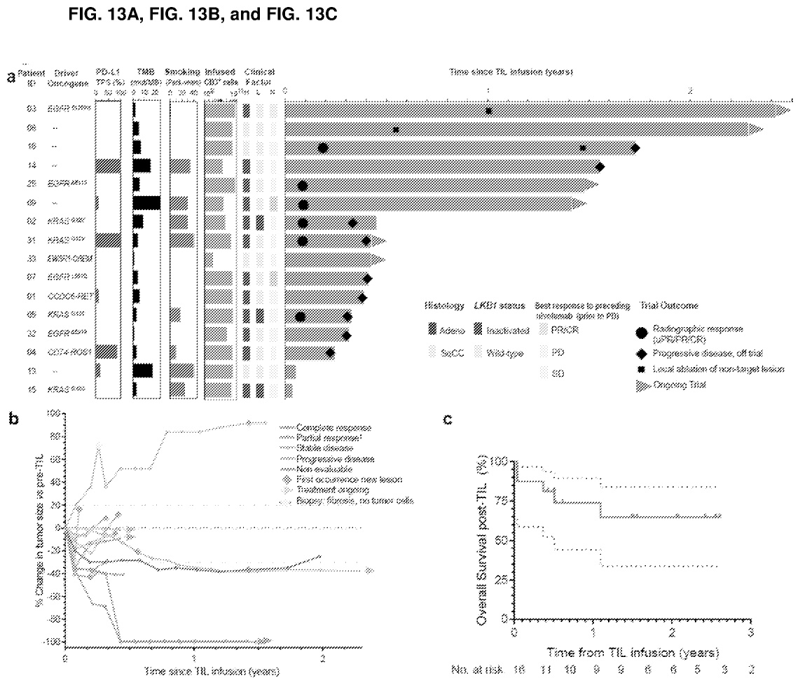

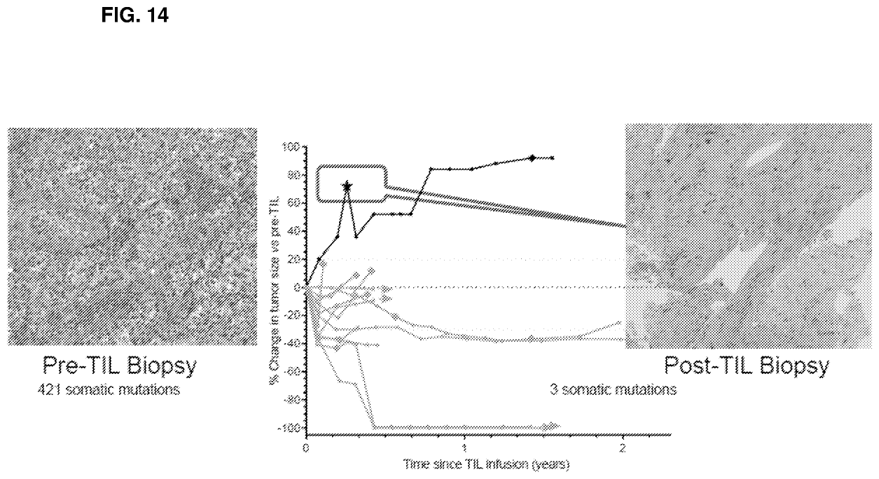

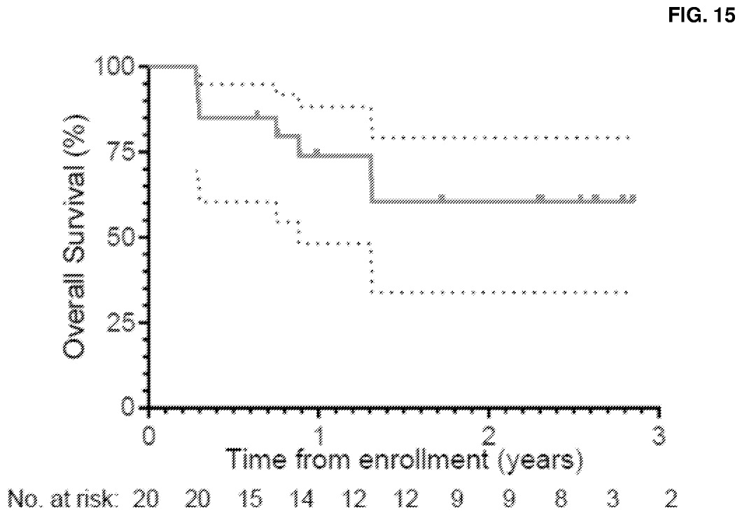

Initial tumor regression occurred in the majority of patients (11 of 16) at the first CT scan performed one month after TIL adoptive cell therapy. Overall, the median best change in sum of target lesion diameters was −35.5% (range +20 to −100). Radiographic response, including unconfirmed response, occurred for 6 of 13 evaluable patients (FIG. 12). These included two complete responses which remain ongoing 1.5 years later (FIG. 13a). Two additional patients had unconfirmed partial response due to subsequent new brain metastases (ID 02, 05). Two additional patients were able to maintain a clinical remission by local ablative therapy of a new ‘escape’ lesion (ID 08, 03) performed between 6 to 17 months post-TIL (FIG. 13b). Another patient (ID 14) had enlargement of her only target lesion, yet biopsy showed only fibrosis (FIG. 14). She remained without disease-related symptoms on trial for 1.5 years, before eventual progression with new lesions. Median survival has not been reached on an intention-to-treat basis (FIG. 15) or post-TIL (FIG. 13c).

(5) Presence of T Cells Recognizing Cancer Mutations

We sequenced whole-exomes and transcriptomes of pretreatment tumors with paired germline and human leukocyte antigen sequencing (Table 4). For each patient, we screened infused T cells for recognition of tumor non-synonymous alterations, including indels and translocations. If aberrant cancer testis antigen was detected, these proteins were also screened. We selected between 13-100% of somatic variants for screening based on RNA coverage and expression. Custom-synthesized peptide pools were pulsed with autologous antigen-presenting cells (APCs). If peptides displaying tumor genomic alterations were confirmed as eliciting T cell reactivity, then these peptides were co-cultured with autologous APCs and T cells over 10 days.

TABLE 4
Correlative assays performed by subject ID.*

| | PD-L1 | Tumor | Tumor | Tumor | TIL | PBMC test | TIL test | Tumor | FEST | IsoCode |
|---|---|---|---|---|---|---|---|---|---|---|
| ID | IHC | WES | RNAseq | TCRseq | Culture | NeoAgs | NeoAgs FMI CDx | | testing | Cytokine |
| 01 | Y | Y | Y | Y | Y | Y | Y | Y | N | Y |
| 02 | Y | Y | Y | Y | Y | Y | N^{§} | Y | Y | Y |
| 03 | Y | Y | Y | Y | Y | Y | Y | Y | N | Y |
| 04 | Y | Y | Y | Y | Y | Y | Y | Y | N | Y |
| 05 | Y | Y | Y | Y | Y | Y | Y | Y | Y | Y |
| 07 | Y | Y | Y | Y | Y | Y | Y | Y | Y | Y |
| 08 | Y | Y | Y | Y | Y | Y | Y | Y | N | Y |
| 09 | Y | Y | Y | Y | Y | Y | Y | Y | Y | Y |
| 12 | Y | Y | Y | Y | Y\hat{ } | Y | Y | Y | N | Y |
| 13 | Y | Y | Y | Y | Y | Y | Y | Y | N | Y |
| 14 | Y | Y | Y | Y | Y | Y | Y | Y | N | Y |
| 15 | Y | Y | Y | Y | Y | Y | Y | Y | N | Y |
| 16 | Y | Y | Y | Y | Y | Y | Y | Y | Y | N |
| 25 | Y | Y | Y | Y | Y | Y | N^{§} | Y | Y | N |
| 26 | Y | Y | Y | Y | Y | Y | Y | Y | N | N |
| 29 | Y | Y | Y | Y | Y | Y | Y | Y | N | N |
| 30 | Y | Y | Y | Y | Y | Y | Y | Y | N | N |
| 31 | Y | Y | Y | Y | Y | Y | Y | Y | Y | N |
| 32 | Y | Y | Y | Y | Y | Y | N^{§} | Y | N | N |
| 33 | Y | Y | Y | Y | Y | Y | N^{§} | Y | N | N |

*Footnotes:
\hat{ }Cultured TIL used for research but insufficient for clinical production.
^{§}For these patients, TIL has too much background
IFN-γ reactivity to interpret ELISpot data.
Abbreviations:
IHC; immunohistochemistry.
WES; whole-exome sequencing.
FEST; Functional Expansion of Specific T Cells³;
FMI, Foundation CDx.

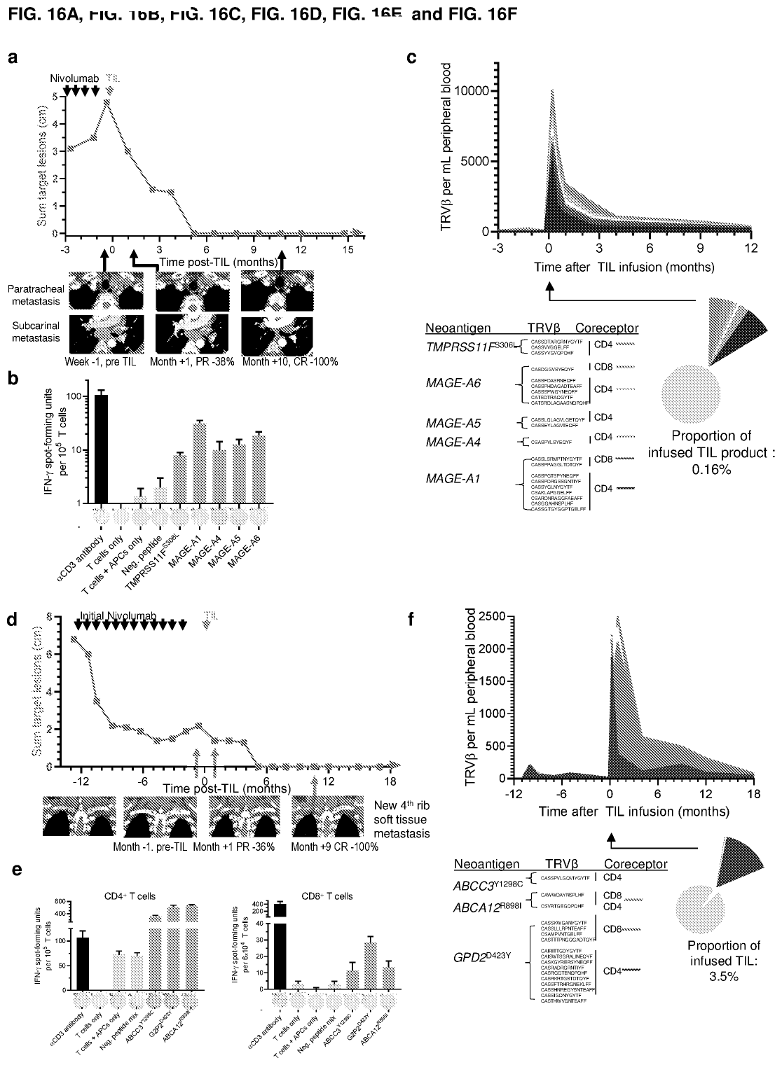

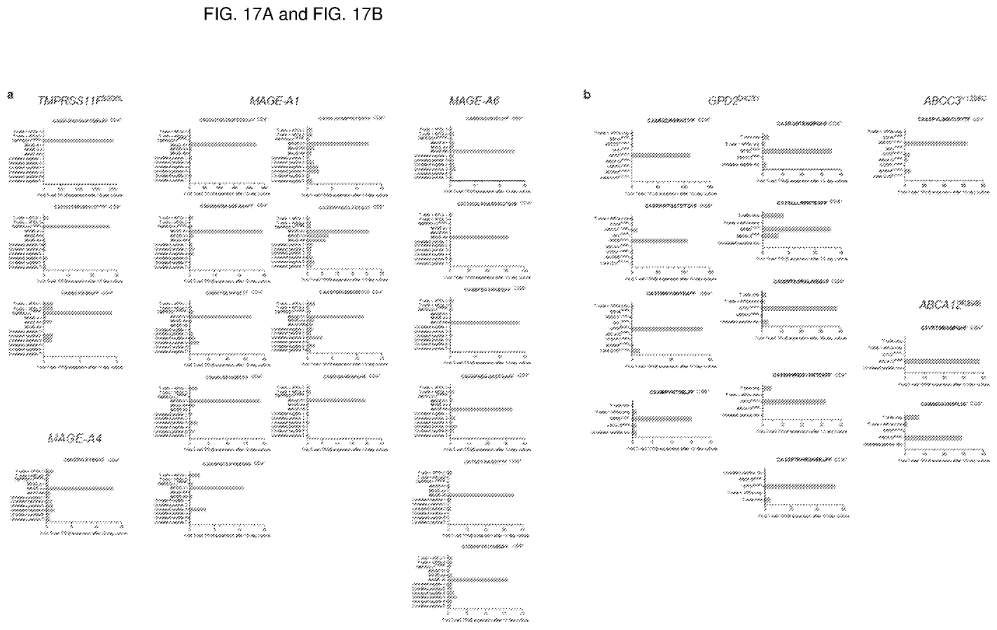

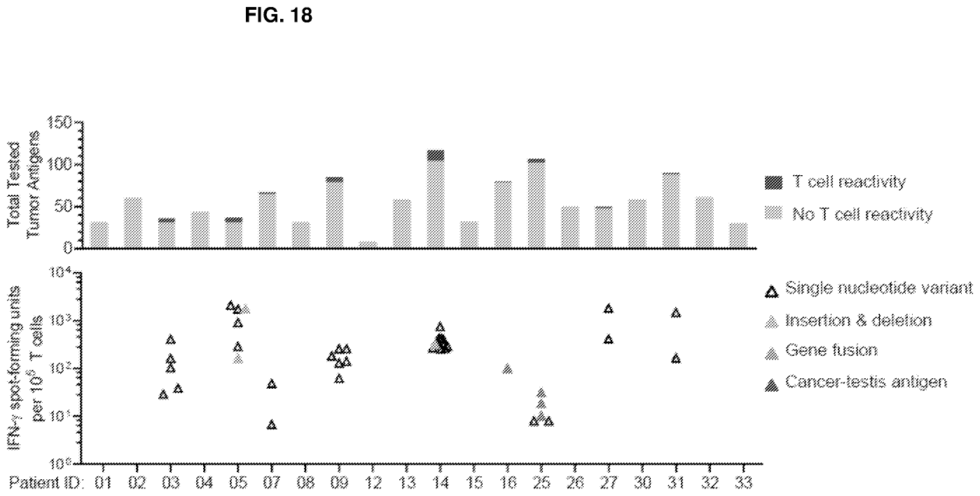

One patient with EGFR^{ΔEx19} lung adenocarcinoma (ID 25) had cancer refractory to nivolumab yet achieved a sustained complete response after TIL treatment (FIG. 16a). Her infused TIL contained not only T clonotypes capable of recognizing one somatic mutation, but also several melanoma associated gene (MAGE) cancer testis antigens discovered to be aberrantly expressed in her tumor (FIG. 16b). Using post-TIL peripheral blood T cells, we used an antigen-recognition method to evaluate in vitro expansion of T-cell clones after peptide stimulation. Nineteen T cell clonotypes exhibited antigen-specific stimulation (FIG. 17a), with mean 140-fold expansion relative to controls. There was a significant increase in these T cell clonotypes circulating in peripheral blood at post-infusion time-points, which persisted over one year (FIG. 16c). Another patient (ID 09) had a complete response to TIL after progressing with a new biopsy-proven soft tissue metastasis while on initial nivolumab (FIG. 16d). Three mutations elicited reactivity from both CD4⁺ and CD8⁺ sorted TIL (FIG. 16e) and peripheral blood T cells. Like Pt 25, a similar surge in antigen-linked T cells clonotypes occurred after TIL infusion within the peripheral blood (FIG. 16f and FIG. 17b). This effect was not evident with nivolumab alone, despite an initial partial response. Another patient (ID 16) with a confirmed partial response had antigens targeting aberrant expression of MAGE-A4. Notably, clinical responses were associated with polyclonal T cell responses against neoantigens derived from a variety of genetic alterations, including single nucleotide variants, insertions/deletions and gene fusions, in addition to cancer testis antigens (FIG. 18). However, specific antigens were not found in some patients. In an exploratory comparison, T cells recognizing aberrant tumor antigens were identified in most patients who achieved uPR, PR, or CR after TIL compared to non-responders (p=0.02, Fishers exact test).

(6) Persistent and Phenotype of T Cells

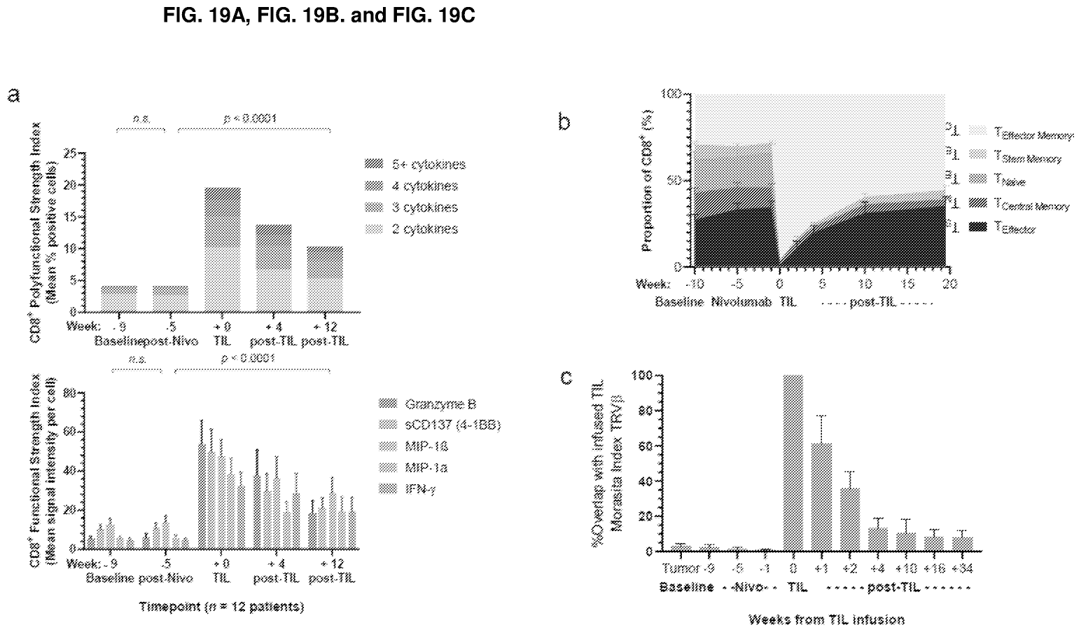

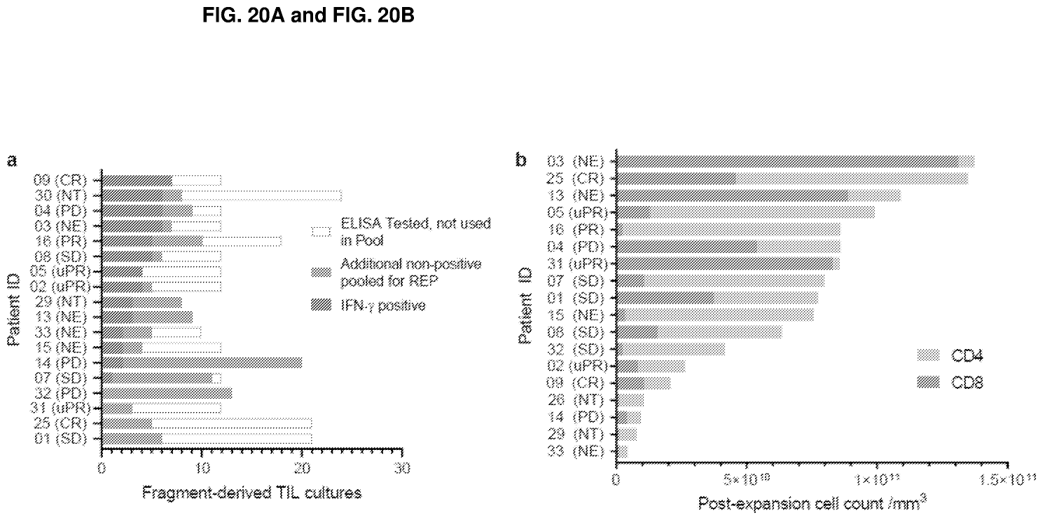

Patients achieved durable conversion of the phenotype of their peripheral T cells after TIL infusion. This effect was not evident from initial nivolumab alone. In particular, the polyfunctional strength index (PSI) of CD8⁺ cells increased at post-infusion timepoints (FIG. 19a). Likewise, peripheral T cells changed to an effector memory (TEM) lineage characteristic of the infused TIL product for CD8⁺ (FIG. 19b) and CD4⁺ cells, followed by gradual recovery of naïve T cell types. In addition, the infused TIL clonotypes largely partially replaced the patients' baseline peripheral repertoire, and then gradually decayed in proportion over the ensuing months (FIG. 19c). No apparent association between TIL clonotype systemic persistence and response was observed. No clear association between response and either cell dose or autologous tumor reactivity was evident (FIG. 20).

(7) Safety and Adverse Events

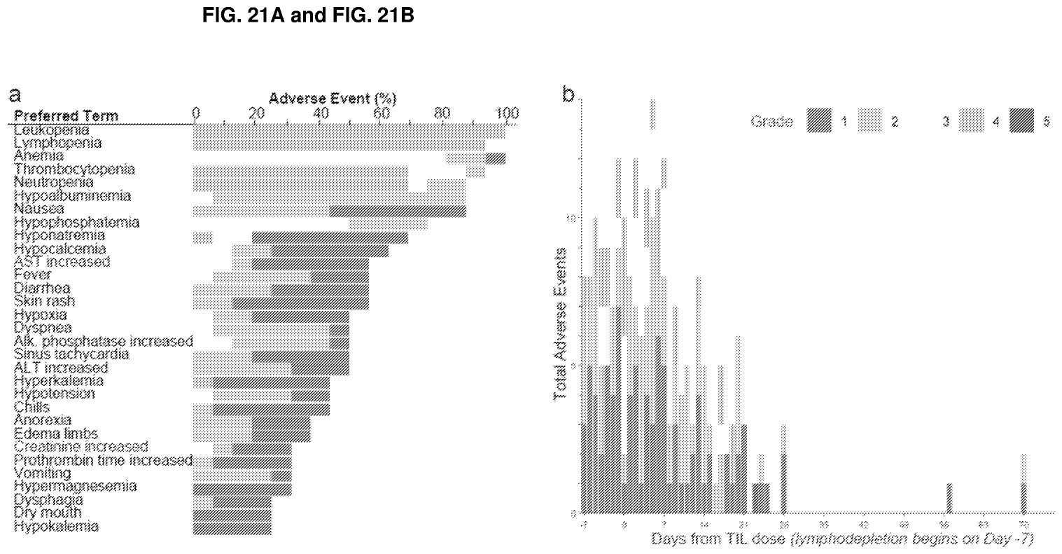

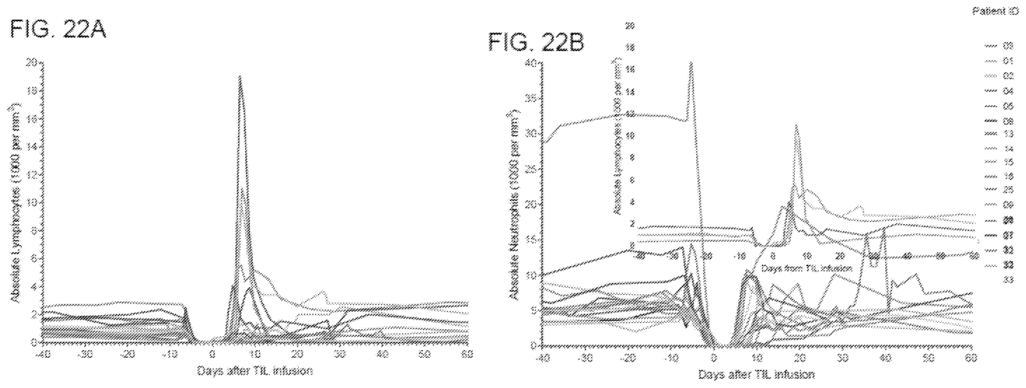

Adverse effects were primarily attributable to the lymphodepletion and interleukin-2 combination (FIG. 21). Common non-hematologic adverse events included hypoalbuminemia, hypophosphatemia, nausea, hyponatremia, and diarrhea. Although manageable for the majority of patients, two patients died prior to a response assessment (ID 13, 15). Both patients had declined to Eastern Cooperative Oncology Group (ECOG) performance status 3 and were requiring supplemental oxygen prior to start of lymphodepletion. Patient 13 was an active smoker with severe right carotid stenosis, and had an ischemic right middle cerebral artery stroke at home at Day +12. Patient 15 was 75 year old limited to wheelchair with metastases replacing 70% of his pulmonary parenchyma. As he continued to decline post-TIL, he transitioned to comfort care without a CT scan. After these patients, stringent pre-lymphodepletion performance status criteria were introduced. Following lymphodepletion, patients recovered lymphoid and myeloid lineages (FIG. 22), with neutrophil count recovering at median 7.5 days (range 4.7 to 20.6). The majority of treatment-emergent adverse events had resolved within one month after TIL. After six months of maintenance nivolumab, one patient had severe thrombocytopenia (ID 16). This event resolved with corticosteroid taper and cessation of nivolumab.

b) Discussion

We found that excisional tumor biopsy and nivolumab followed by administration of cyclophosphamide, fludarabine, IL-2 with TIL infusion in pretreated metastatic lung cancer was feasible, and had manageable adverse effects. We observed that lymphocytes could be successfully expanded from most patients' tumors, and were largely capable of autologous tumor recognition. In addition to two durable complete responses, we also observed some patients who derived clinical benefit in a variety of other ways, including local ablation of a single metachronous new lesion. One durable complete response occurred in a TMB_{low}, PD-L1_{negative} never smoker, who was refractory to nivolumab. This is particularly encouraging for the large subset of never-smoker patients, for whom immune checkpoint inhibitors have historically had limited efficacy. Together with results in breast cancer, the data indicate that TIL can mediate effective responses in tumor subtypes which are not sensitive to traditional immune checkpoint targeted therapy. Therefore, therapy with TIL can extend the scope and impact of immunotherapy into wider populations.

This report adds to growing evidence that TIL adoptive cell therapy can be active in a variety of epithelial malignancies. In contrast to prior experience in melanoma, we found a high proportion of CD4⁺TIL in NSCLC. Despite this, CD4⁺ predominant TIL were capable of mediating responses, and antigen-specific CD4 clones recognizing immunogenic tumor mutations were identified (FIG. 16). Antigen-specific CD4 cells recognizing mutations such as B_{RAF}^{V600E} have previously been identified in melanoma, and have mediated tumor regressions in gastrointestinal malignancies. Therefore, an important requirement for TIL efficacy is the presence of sufficient T cells which recognize immunogenic genomic alterations. In this case, we found autologous T cells reactive to against multiple types of tumor antigens (FIG. 18). Further study is needed to determine the threshold polyclonality, or diversity of antigens targeted by an infused TIL repertoire, required to achieve a durable clinical response.

Lymphodepletion chemotherapy and IL-2 are prerequisite to ensure homeostatic expansion and engraftment of the infused TIL. This incumbent toxicity, together with the required production infrastructure, has historically limited experimentation with TIL to select centers. We had 2 patients with early deaths related to poor performance status and inanition caused by rapidly progressive disease, combined with physiologic stress of lymphodepletion and IL-2. By the time TIL were manufactured, these patients were infirm and requiring supplemental oxygen. Future strategies to expedite TIL manufacturing and curtail the physiologic footprint of lymphodepletion may be needed to ensure the broader adoption of TIL in the lung cancer community. To this end, newer, less invasive approaches to isolate and expansion tumor neoepitope reactive T cells are being pioneered to expand to indications with limited tumor access. For example, PD-1⁺ selection can enrich for neoantigen-reactive T cells from the blood of patients with melanoma and gastrointestinal cancer. Likewise, sorting for memory T cell markers can capture most of the mutated neoantigen-reactive T cells in the peripheral blood of epithelial cancer patients.

This study was characterized by important design limitations. Within this combination therapy, the individual component contributions of IL-2, cyclophosphamide, fludarabine, and nivolumab are difficult to assess. Neither lymphodepletion nor IL-2 monotherapy have historically reported durable responses in NSCLC. We chose to exclusively manufacture TIL from anti-PD(L)-1 treatment naïve tumors. Exhausted TIL clones have limited ability for phenotype transition to an activated state after PD(L)-1 blockade. Therefore pre-treatment tumor harvest followed by PD-1 blockade represents the most ideal scenario to test TIL efficacy, while still controlling for the effect of nivolumab monotherapy. In particular, one patient (ID 09) still had a complete response to TIL after suffering relapse after 1 year on nivolumab. This indicates that anti-PD-1 treatment-naive TIL can still mediate specificity and cytotoxicity against PD-1-experienced tumor cells. Based on its activity and safety profile, TIL is a rational therapy to further investigate for fit, motivated NSCLC patients who have progressed on prior PD-(L)1 based treatment. Larger trials are required to further define the optimal biomarkers of response.

c) Methods

(1) Patients

From October 2017 to January 2020, we enrolled patients in a dual center phase I investigator-initiated trial designed to test the feasibility of adoptive cell therapy with TIL (ClinicalTrials.gov NCT03215810). Eligible patients were adults with histologically proven stage 4 NSCLC who were not candidates for surgical resection for curative intent. Patients were required to have an accessible metastasis to procure for TIL with acceptable anticipated perioperative risk, and also at least one separate additional measurable tumor lesion on computed tomography (CT). All patients were required to have received fewer than six prior lines of systemic therapy. Prior therapy could not have included a programmed death 1 (PD-1) or PD-L1 inhibitor. Patients were required to have sufficient cardiopulmonary function, including a cardiac stress test showing no reversible ischemia. Patients with active brain metastases, autoimmune conditions, or acquired immunodeficiency were excluded.

Patients with epidermal growth factor receptor (EGFR) mutation or anaplastic lymphoma kinase (ALK) translocation were allowed if they had progressed on ≥1 prior approved tyrosine kinase inhibitor (TKI). Comprehensive exome sequencing was performed on the resected tumor obtained after enrollment. This step revealed an uncommon EGFR^{L861Q} in Patient 07 and a variant EML4-ALK in Patient 12 which was not detected on cell-free DNA assay prior to enrollment.

(2) Study Conduct

This study was conducted at two academic medical centers in the United States. The primary endpoints were safety and feasibility. Key secondary endpoints were radiologic response rate and overall survival. Adverse events were recorded according to National Cancer Institute Common Terminology Criteria for Adverse Events, v4.0. Tumors were assessed per Response Evaluation Criteria in Solid Tumors (RECIST), version 1.1. Patients were deemed eligible and enrolled after passing all screening procedures, prior to tumor resection.

(3) Study Design

After excisional biopsy of the metastasis, patients were treated with 4 cycles of intravenous nivolumab at a dose of 240 mg every 2 weeks (FIG. 9a). If patients had evidence of clinical benefit after two sequential CT scans, then nivolumab was continued until progression. If there was evidence of progression, as defined by tumor enlargement or new lesions, then we proceeded to administer lymphodepletion and TIL treatment. TIL infusion was defined as Day 0. Patients received lymphodepletion intravenously with cyclophosphamide at a dose of 60 mg per kg daily for Day −7 and −6 with fludarabine daily at a dose of 30 mg per m² of body surface area for Days −7 to −3. Patients were then admitted to the hospital and received TIL infusion on Day 0, followed by continuous aldesleukin infusion beginning 12 hours later at a dose of 18 million international units (MIU) per m² over 6, then 12, and then 24 hours followed by 4.5 MIU/m² over 24 hours for 3 consecutive days. Patients were discharged once they had >1000 k/μL peripheral neutrophils. CT scans were performed every 6 weeks and leukapheresis at Week 4 to collect immune cells for functional studies. Then patients resumed nivolumab 480 mg every 4 weeks for up to 1 year. If patients had a single site of progressive tumor, this was formally recorded as progression. However, these patients were permitted to remain on trial after successful local ablative therapy using either resection or stereotactic radiation. Oral prophylaxis for neumocystis and herpes simplex virus was given for 6 months after TIL treatment.

(4) Study Supervision

This study was approved by Institutional Review Board (FWA 0023875) and underwent FDA safety review (IND 17489). Written informed consent was obtained, including germline DNA sequencing. The trial was designed and reported by the authors, who assure the accuracy of the data. Only the authors participated in manuscript preparation. The trial was primarily supported by Stand Up to Cancer Foundation. Nivolumab was supplied by E. R. Squibb & Sons LLC. Aldesleukin (IL-2) was supplied by Clinigen Group, Inc. Funding to the Moffitt Cell Therapies Facility was provided by Iovance Biotherapeutics, Inc. The companies played no other role in the study or report.

(5) TIL Product Processing, Selection and Expansion

The majority of harvested metastases were pleural nodules or supraclavicular lymph nodes. Freshly resected patient tumors were dissected by the surgeon in a sterile back table of the operating room and adventitial tissue removed. Tumor was then placed in sterile RPMI media container and delivered to the Moffitt Cell Therapy Facility. Tumors were processed within 3 to 12 hours of receipt by a cell technician within an ISO 7 cGMP certified clean room. Tumor fragments were minced into 3-mm cubes. Each tumor was cut into 48 fragments. Tumor fragments were placed individually into two separate 24-well culture plates in media supplemented with 600 IU/mL of IL-2 and 10% human AB serum. Fragments were surveyed for proliferation every 2-3 days for up to 5 weeks. TIL were expanded to a new well of a 24-well plate when they became ≥80% confluent. TIL derived from each fragment was kept separate. Media containing 6,000-IU/mL IL-2 was supplemented every 3-4 days to maintain TIL growth.

T cells were screened for specificity to autologous tumor by incubation with a suspension of tumor cells and interferon-gamma (IFN-γ) quantification. For autologous reactivity testing, a portion of excess tumor was disaggregated into a single-cell suspension to create substrate tumor for enzyme linked immunoassays (ELISA). Tumor was minced and carefully mixed in digestion media with collagenase (type II and type IV), hyaluronidase, and DNAase (Fisher Scientific Co., Pittsburgh, Pa., USA). Then undigested tumor was filtered from the cell suspension to generate a single-cell suspension. Intact cells were enriched with a Ficoll-Hypaque (GE Healthcare Bio-Sciences, Pittsburgh, Pa., USA) gradient, and viable tumor cells were enumerated by trypan blue exclusion. Lymphocytes from fragment wells with the most pronounced proliferation were tested for autologous tumor reactivity by overnight co-culture with autologous and/or HLA-matched and HLA-mismatched tumor suspension in a 1:1 ratio. IFN-γ within the supernatants was detected by ELISA. Autologous reactivity was judged based on at least twofold higher IFN-γ compared with HLA-mismatched tumor cells. These TIL pools containing the reactive TIL were selected, pooled and cryopreserved for later rapid expansion protocol (REP). If insufficient or no autologously reactive TIL were identified, then additional fragments were used based on their growth rate.

Positive cultures were pooled and cryopreserved for each patient. If patients had progressive disease on nivolumab, then TIL were thawed and expanded in CD3 antibody, irradiated allogeneic feeder cells, and IL2 over 14 days. For REP, TIL were cultured in in G-Rex 100 MCS flasks and incubated at 37.0 and 5% CO2 at a 1:200 ratio with a layer of irradiated allogeneic donor feeder cells, and IL-2 at a concentration of 3,000 IU/mL and anti-CD3 (Ortho Biotech) at a concentration of 30 ng/mL were added to the flasks. Flasks were incubated at 37.0 and monitored for the next 7 days and split as needed to maintain the TIL concentration at 2×10⁶/mL. Then cells were collected, washed, and concentrated to 1.0 L or less. Cell viability was tested using acridine orange (AO) and propidium iodide (PI) dyes. Total live cells were counted using a Cellometer Auto 2000 (Nexcelom Bioscience, Lawrence, Mass., USA). The final product was tested for sterility and then gravity infused into the patient at a rate of approximately 300 mL per hour. To characterize the final product, cells from were stained with 7-AAD, CD3-FITC, CD4-PE, CD45-V500, and CD8-APC. Flow cytometry data was collected using a FACSCanto (BD Biosciences, San Jose, Calif., USA) and FlowJo Software (Treestar, Ashland, Oreg., USA). Upon harvest, TIL product underwent Quality Control testing and had to meet the following criteria prior to release for patient administration: ≥45% CD45+ by flow cytometry, <5EU/Kg of endotoxin, no detectable mycoplasma, negative gram stain, ≥70% viability and sterile blood culture from day −3 of REP. The TIL were also assessed by flow cytometry for CD8 (mean=57.27±33.77%) and CD4 (mean=41.00±32.86%) composition.

(6) Flow Cytometry of Lymphocytes

Human peripheral blood samples were collected in 4 heparin tubes at baseline, following nivolumab infusion and multiple timepoints following TIL infusion. Peripheral blood mononuclear cells (PBMC) were collected using a Ficoll gradient and cryopreserved in 10% DMSO and FBS. Cells were thawed in media, and subsequently stained in PBS containing 5% FBS (vol/vol, FACS buffer) with: CD3 BUV496, CD56 BUV563, CD4 BUV737, CD197 BV421, CD28 BV480, CD14 BV605, CD19 BV605, CD95 BV711, CD195 BV786, CD127 PE, CD194 PE-Cy7, CD45RA Alexa488, CD25 PerCP-Cy5.5, NKG2D APC, Tim3 BV421, PD1 BV480, CD226 BV711, CTLA4 BV786, Lag3 PE, TIGIT PE-Cy7, CD244 Alexa488, CD27 PerCP-Cy5.5, BTLA APC from BD Biosciences. Dead cells were excluded using the Zombie NIR Fixable Viability Kit from Biolegend, incubated at 4° C. for 1 hr, then washed twice with FACS buffer, and finally fixed in PBS containing 1% paraformaldehyde before running flow cytometry. Cells were acquired on a BD FACSymphony™ A5, and data were analyzed with FlowJo Version 10.0 software.

(7) Polyfunctional Strength Index

Polyfunctional strength index (PSI) measures the ability of single CD3⁺ cells to secrete multiple different cytokines after stimulation. CD8⁺ T cells were sorted from TIL and peripheral blood, and stimulated with CD28 and CD3 antibody. Testing was performed using the Isoplexis IsoCode chip using the 32 human cytokine single-cell proteome panel. Pre- and post-infusion timepoints were compared using a mixed-effects model (REML) with stacked matching with Geisser-Greenhouse correction, in an exploratory comparison. Multiplicity adjusted p value was performed using Dunnett's control.

(8) Generation of Autologous Dendritic Cells

The plastic adherence method was used to generate monocyte-derived dendritic cells. Autologous PBMCs or apheresis samples were thawed and resuspended at 2-5·10⁶/mL with AIM-V media (Life Technologies). The cells were incubated in a tissue culture flask of an appropriate size at 37° C., 5% CO₂. After 90 minutes, non-adherent cells were collected and the flasks were washed with AIM-V media twice with an interval of 60 minutes, after which DC media were added. DC media were made from RPMI 1640 containing 5% human serum (Sigma), 100 U/ml penicillin and 100 μg/ml streptomycin, 2 mM L-glutamine (media supplements were from Life Technologies), 800 IU/ml GM-CSF and 800 U/ml IL-4 (cytokines were from Peprotech). On day 3, fresh DC media was supplemented. Dendritic cells were harvested on day 5-7 and frozen in 10% DMSO for co-culture experiments.

(9) Screening T Cells for Reactivity to Antigens Using the ELISpot Assay

Both effector T cells & DCs were pre-thawed 24 hours before co-culture in IL-2 free media. In the ELISpot assay, PVDF membrane plates (Millipore, MAIPSWU10) were pre-activated with 50 μl 70% ethanol per well for 2 min and washed thrice with PBS. Then 50 μl purified IFN-γ capture antibody (Mabtech, clone: 1-D1K) were added per well for incubation at 4° C. overnight. Before co-culture, wells were washed thrice with PBS and incubated with 100 μl AIM-V media at room temperature for 1 hour. In co-culture, when using TIL as effector T cells, 1·10⁴ to 3·10⁴ T cells were placed per well in a 96-well flat bottom plate. When T cells isolated from apheresis samples (4 weeks post TIL infusion) were used as effector cells, 1·10⁵ cells were used per well in a 96-well flat bottom plate. Effector T cells were co-cultured with 5·10³-1·10⁴ DCs cells loading 1-10 μg/mL tumor antigen peptides. Cytomegalovirus, Epstein-Barr, or Hepatitis (CEF) viral peptides and/or plate-bound OKT3 (1 μg/mL) were used as positive controls. Effector T cells only and/or effector T cells co-cultured with unloaded DCs were used as negative controls.

After 24 hours of co-culture, the plate was washed 6× with PBS plus 0.05% Tween-20 and then incubated with 100 μl per well of 0.22 μm filtered 1 μg/mL biotinylated anti-IFN-γ detection antibody (Mabtech, clone: 7-B6-1) for 2 hours. Another 4× wash with PBS-T was performed and 100 μl per well of streptavidin-ALP (Mabtech, diluted 1:1000) were added for a 1-hour incubation. The plate was washed 6× with PBS and developed with 100 μl per well of 0.45 μm filtered BCIP/NBT substrate solution (KPL, Inc.) for 10 minutes. Finally, the plate was washed thoroughly with cold tap water and then scanned and auto-counted using an ImmunoSpot ELISpot plate reader (Cellular Technologies, Ltd).

(10) Identification of Tumor Antigen Specific TCR Clonotypes (FEST)

The protocol to identify tumor antigen specific TCR clonotypes followed the published MANAFEST assay with modifications. Apheresis samples (4 weeks post TIL infusion) were thawed and T cells were isolated using EasySep Human T cell Enrichment Kit (Stemcell Technologies). Then T cells were washed and resuspended at 2·10⁶/mL in AIM-V media. Dendritic cells were thawed and resuspended at 5·10⁴/mL in AIM-V media. After identifying T cell-recognized tumor antigens, 125 μl T cells were co-cultured with 125 μl DCs in a 96-well round bottom plate and tumor antigen proteins/peptides were added at the concentration of 1-10 μg/mL. On day 3, supernatants were half replaced with fresh AIM-V media containing 100 IU/mL IL-2, 50 ng/mL IL-7 and 50 ng/mL IL-15. On day 7, supernatants were half replaced with fresh AIM-V media containing 200 IU/mL IL-2, 50 ng/mL IL-7 and 50 ng/mL IL-15. Cell cultures were harvested on day 10 for DNA extraction (Qiagen) and further TCRVβ sequencing. Analysis of TCRseq data to figure out tumor antigen specific expansion of TCR clonotypes was performed.

The absolute number of T cell clonotypes in the peripheral blood was calculated by using the absolute lymphocyte count derived from automated cell differential provided by the medical laboratory, multiplied by the percentage of CD3⁺ gated of all lymphocytes by flow cytometry, multiplied by the frequency of this specific clone as a proportion of all productive rearrangements derived from immunoSEQ TRVβ DNA sequencing of that sample.

(11) Analysis of Tumor Markers

Resected baseline tumors were stained for PD-L1 using 22C3 pharmDx immunohistochemistry antibody and scored using tumor proportion score (TPS) by a trained pathologist. Tumor mutation burden (TMB) was estimated using targeted exome sequencing from Foundation Medicine. All tumor marker inferences were defined as exploratory in the study protocol.

(12) Nucleic Acid Extraction

Portions of the same tumor resected for TIL were snap-frozen in liquid nitrogen and stored at −80 C. In batches, tumor sections were selected based on H&E sections using laser-capture macrodissection (LCMD) and pulverized into aliquots of 15 mg. Aliquots were extracted for RNA and DNA using Allprep kits (Qiagen). DNA and RNA was quantified using Invitrogen Qubit Fluorometer, purity assessed using Thermo Scientific™ NanoDrop and integrity assessed with Agilent 4200 TapeStation system. Mononuclear cells were isolated from peripheral blood collected in EDTA tubes, using a Ficoll gradient. Cell pellets were snap-frozen, and later DNA was extracted in water using mini-prep kit (Qiagen). Quality metrics were assessed as described above.

(13) Whole Exome Sequencing

Whole exome sequencing was performed in order to identify somatic mutations in DNA extracted from pre-treatment tumor tissue. Two hundred ng of DNA was used as input into the Agilent SureSelect XT Clinical Research Exome kit, which includes the exon targets of Agilent's v5 whole-exome kit, with increased coverage at 5000 disease-associated targets. Briefly, for each tumor DNA sample and germline DNA sample, a genomic DNA library was constructed according to the manufacturer's protocol and the size and quality of the library was evaluated using the Agilent BioAnalyzer. An equimolar amount of library DNA was used for a whole-exome enrichment using the Agilent capture baits and after quantitative PCR library quantitation and quality control analysis on the BioAnalyzer, 75-base paired-end sequences were generated using v2 chemistry on an Illumina NextSeq 500 sequencer.

Human Leukocyte Antigen (HLA) Class I and II locus sequencing was performed in the Moffitt HLA laboratory (ASHI accreditation 07-3-FL-18-1). DNA from nucleated peripheral blood cells underwent QC using Nanodrop followed by next generation sequencing using the MIA FORA FLEX HLA Typing Kit (Immucor, Norcross Ga.). A confirmatory sequence-specific oligonucleotide (SSO) DNA typing (LABType, One Lambda) was performed to resolve ambiguous alleles.

(14) Transcriptome Sequencing

RNA-sequencing libraries were prepared from RNA extracted from pre-treatment tumor tissue using the NuGen FFPE RNA-Seq Multiplex System (Tecan US, Inc., Morrisville, N.C.). Briefly, 50 ng of DNase-treated RNA was used to generate cDNA and a strand-specific library following the manufacturer's protocol. Library molecules containing ribosomal RNA sequences were depleted using the NuGen AnyDeplete probe-based enzymatic process. The final libraries were assessed for quality on the Agilent TapeStation (Agilent Technologies, Inc., Wilmington Del.), and quantitative RT-PCR for library quantification was performed using the Kapa Library Quantification Kit (Roche Sequencing, Pleasanton, Calif.). The libraries were sequenced on the Illumina NextSeq 500 sequencer with a 75-base paired-end run in order to generate 80-100 million read pairs per sample.

(15) T Cell Repertoire Sequencing

T-cell receptor repertoire analysis was performed using Adaptive Biotechnologies immunoSEQ v3 assay, which employs bias-controlled multiplex PCR amplification and high-throughput sequencing to target rearranged T-cell receptor genes. The manufacturer's protocol was followed in order to utilize the immunoSEQ hsTCRB kit to amplify the complementarity determining region 3 (CDR3) locus from genomic DNA extracted from sorted T-cells or tissue. Following the confirmation of amplification and a successful final library preparation, sequencing was performed on the Illumina NextSeq 500 to a depth of 2 million pairs of sequencing reads per sample for survey-level analysis, or 5-6 million pairs per sample for deep-level analysis. The data were then analyzed using the AdaptiveBiotechnologies immunoSEQ Analyzer software, which identifies and counts the V, D, and J genes, filters non-productive sequences, and reports and tracks T cell clonality.

(16) Proteomics

Tissue samples were pulverized and denaturing buffer was used to extract the proteins, followed by protein reduction, alkylation and trypsin digestion. The tryptic peptides were acidified and desalted with C18 cartridge. After lyophilization, the peptides were fractionated off-line with basic pH reversed phase HPLC and 24 concatenated fractions were collected. LC-MS/MS analysis of each fraction was performed with a nanoflow ultra high performance liquid chromatograph (RSLC, Dionex, Sunnyvale, Calif.) coupled to an electrospray bench top orbitrap mass spectrometer (Q-Exactive plus, Thermo, San Jose, Calif.). The result was analyzed with MaxQuant software for protein identification and label free quantitation.

(17) Identification of Somatic Mutations

Somatic mutations were identified from whole-exome sequence reads and aligned to the reference human genome (hg19) with the Burrows-Wheeler Aligner (BWA). Insertion/deletion realignment and quality score recalibration were performed with the Genome Analysis ToolKit. Tumor specific mutations were identified with Strelka and MuTect, and were annotated to determine genic context, including non-synonymous, missense, splicing calls, using ANNOVAR. Only somatic mutations predicted by both algorithms were included for subsequent neoantigen prediction. Additional contextual information was incorporated, including allele frequency was derived from available resources including 1000 Genomes, the NHLBI Exome Sequence Project, and the Exome Aggregation Consortium, in silico function impact predictions, and observed impacts from databases including ClinVar and the Collection Of Somatic Mutations In Cancer (COSMIC).

(18) Gene Expression and Fusion Detection

Sequence reads were aligned to the human reference genome in a splice-aware fashion using STAR, allowing for accurate alignments of sequences across introns. Aligned sequences were assigned to exons using the HTseq package. DESeq2 was used for normalization of gene expression. Aberrant cancer testis antigen (MAGE) expression was identified based on gene expression level and verified using the sum of peptide intensities for the corresponding fasta protein proteomic sequencing described above. Gene fusions were identified from RNAseq data using FusionCatcher.

(19) Neoantigen Prediction and Prioritization

Neoantigens can be identified by extracting altered peptides from mutation data with ANNOVAR or directly from FusionCatcher results, and then predicting MHC binding against patient-specific HLA type using NetMHC NetMHCpan and NetMHCIIpan as implemented by the Immune Epitope Database. HLA types were derived from clinical HLA laboratory sequencing data. Neoantigens were prioritized by a combination of predicted MHC binding affinity, variant allele frequency, RNA expression of the genes where the mutation is potentially located, and evidence of reads carrying the mutated base.

(20) Statistical Analysis

Adverse events were continuously monitored. Acceptable safety was prospectively defined as a dose-limiting toxicity rate of 17% or less, using a Pocock-type stopping boundary with continuous monitoring. Dose-limiting toxicity was defined as grade ≥4 toxicity with attribution to ACT. Baseline tumor measurements were defined on Week-1 CT prior to TIL, with subsequent assessment every 6 weeks. Overall survival was calculated using the Kaplan-Meier method, and 95% confidence interval included for medians and curves. Pathological, genomic, and functional immunologic testing was performed and analyzed as described. Reported p values were two-sided with a significance of 0.05, unless otherwise noted. Normality was not assumed, and non-parametric tests were performed where applicable. For functional expansion of specific T-cells (FEST) analysis, a threshold p-value of 0.05 was adjusted by Benjamini-Hochberg procedure to control for multiple comparisons.

### D. References

Anders, S. & Huber, W. Differential expression analysis for sequence count data. Nature Precedings, 1-1 (2010).
Anders, S., Pyl, P. T. & Huber, W. HTSeq—a Python framework to work with high-throughput sequencing data. Bioinformatics 31, 166-169 (2015).
Andersen, R., et al. Long-lasting complete responses in patients with metastatic melanoma after adoptive cell therapy with tumor-infiltrating lymphocytes and an attenuated IL2 regimen. Clinical Cancer Research 22, 3734-3745 (2016).
Cafri, G., et al. Memory T cells targeting oncogenic mutations detected in peripheral blood of epithelial cancer patients. Nature communications 10, 1-9 (2019).
Cibulskis, K., et al. Sensitive detection of somatic point mutations in impure and heterogeneous cancer samples. Nature biotechnology 31, 213-219 (2013).
Danilova, L., et al. The Mutation-Associated Neoantigen Functional Expansion of Specific T Cells (MANAFEST) Assay: A Sensitive Platform for Monitoring Antitumor Immunity. Cancer Immunol Res 6, 888-899 (2018).
DePristo, M. A., et al. A framework for variation discovery and genotyping using next-generation DNA sequencing data. Nature genetics 43, 491 (2011).
Dobin, A., et al. STAR: ultrafast universal RNA-seq aligner. Bioinformatics 29, 15-21 (2013).
Forde, P. M., et al. Neoadjuvant PD-1 blockade in resectable lung cancer. New England Journal of Medicine 378, 1976-1986 (2018).
Gainor, J. F., et al. EGFR mutations and ALK rearrangements are associated with low response rates to PD-1 pathway blockade in non-small cell lung cancer: a retrospective analysis. Clinical cancer research 22, 4585-4593 (2016).
Gros, A., et al. Recognition of human gastrointestinal cancer neoantigens by circulating PD-1+ lymphocytes. The Journal of clinical investigation 129(2019).
Ivanova, A., Qaqish, B. F. & Schell, M. J. Continuous toxicity monitoring in phase II trials in oncology. Biometrics 61, 540-545 (2005).
Jurtz, V., et al. NetMHCpan-4.0: improved peptide—MHC class I interaction predictions integrating eluted ligand and peptide binding affinity data. The Journal of Immunology 199, 3360-3368 (2017).
Katz, S. I., et al. Radiologic Pseudoprogression during Anti-PD-1 Therapy for Advanced Non-Small Cell Lung Cancer. Journal of Thoracic Oncology 13, 978-986 (2018).
Lek, M., et al. Analysis of protein-coding genetic variation in 60,706 humans. Nature 536, 285-291 (2016).
Li, H. & Durbin, R. Fast and accurate short read alignment with Burrows-Wheeler transform. bioinformatics 25, 1754-1760 (2009).
Lopes, G., et al. Pembrolizumab (pembro) versus platinum-based chemotherapy (chemo) as first-line therapy for advanced/metastatic NSCLC with a PD-L1 tumor proportion score (TPS)≥1%: open-label, phase 3 KEYNOTE-042 study. (American Society of Clinical Oncology, 2018).
Malekzadeh, P., et al. Antigen experienced T cells from peripheral blood recognize p53 neoantigens. Clinical Cancer Research 26, 1267-1276 (2020).
Nielsen, M., et al. Reliable prediction of T-cell epitopes using neural networks with novel sequence representations. Protein Science 12, 1007-1017 (2003).
Precopio, M. L., et al. Immunization with vaccinia virus induces polyfunctional and phenotypically distinctive CD8⁺ T cell responses. The Journal of experimental medicine 204, 1405-1416 (2007).
Renkvist, N., Castelli, C., Robbins, P. F. & Parmiani, G. A listing of human tumor antigens recognized by T cells. Cancer Immunology, Immunotherapy 50, 3-15 (2001).
Reynisson, B., et al. Improved prediction of MHC II antigen presentation through integration and motif deconvolution of mass spectrometry MHC eluted ligand data. Journal of Proteome Research (2020).
Rosenberg, S. A., et al. Durable complete responses in heavily pretreated patients with metastatic melanoma using T-cell transfer immunotherapy. Clinical Cancer Research 17, 4550-4557 (2011).
Saunders, C. T., et al. Strelka: accurate somatic small-variant calling from sequenced tumor-normal sample pairs. Bioinformatics 28, 1811-1817 (2012).
Simpson-Abelson, M., D'Arigo, K., Hilton, F., Fardis, M. & Chartier, C. 1053P Iovance generation-2 tumour-infiltrating lymphocyte (TIL) product is reinvigorated during the manufacturing process. Annals of Oncology 31, 5720 (2020).
Stevanović, S., et al. Complete regression of metastatic cervical cancer after treatment with human papillomavirus-targeted tumor-infiltrating T cells. Journal of Clinical Oncology 33, 1543 (2015).
Tran, E., et al. Cancer immunotherapy based on mutation-specific CD4+ T cells in a patient with epithelial cancer. Science 344, 641-645 (2014).
Tran, E., et al. Immunogenicity of somatic mutations in human gastrointestinal cancers. Science 350, 1387-1390 (2015).
Tran, E., et al. T-cell transfer therapy targeting mutant KRAS in cancer. New England Journal of Medicine 375, 2255-2262 (2016).
Veatch, J. R., et al. Tumor-infiltrating BRAF V600E-specific CD4+ T cells correlated with complete clinical response in melanoma. The Journal of clinical investigation 128, 1563-1568 (2018).
Vita, R., et al. The immune epitope database (IEDB): 2018 update. Nucleic acids research 47, D339-D343 (2019).
Wang, K., Li, M. & Hakonarson, H. ANNOVAR: functional annotation of genetic variants from high-throughput sequencing data. Nucleic acids research 38, e164-e164 (2010).
Weber, J. S., et al. Safety profile of nivolumab monotherapy: a pooled analysis of patients with advanced melanoma. (2017).
Xue, Q., et al. Single-cell multiplexed cytokine profiling of CD19 CAR-T cells reveals a diverse landscape of polyfunctional antigen-specific response. Journal for immunotherapy of cancer 5, 85 (2017).
Yeh, S., et al. Ocular and systemic autoimmunity after successful tumor-infiltrating lymphocyte immunotherapy for recurrent, metastatic melanoma. Ophthalmology 116, 981-989. e981 (2009).
Yost, K. E., et al. Clonal replacement of tumor-specific T cells following PD-1 blockade. Nature medicine 25, 1251-1259 (2019).
Zacharakis, N., et al Immune recognition of somatic mutations leading to complete durable regression in metastatic breast cancer. Nature medicine 24, 724-730 (2018).

1. A T cell receptor that recognizes for one or more neoantigens.

2. The TCR of claim 1, wherein the neoantigen recognized by the TCR comprises the sequence DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38).

3. The TCR of claim 1, wherein the TCR comprises the sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), CASSDTARGRNYGYTF (SEQ ID NO: 39), CASSVVGGELFF (SEQ ID NO: 40), CASSPYGTESGANVLTF (SEQ ID NO: 41), CASSQDFRDRAGELFF (SEQ ID NO: 42), CASSQDPSGSYEQYF (SEQ ID NO: 43), CASSQEVGGYTF (SEQ ID NO: 44), CASSQVPGSYEQYF (SEQ ID NO: 45), CSATGTKTNYGYTF (SEQ ID NO: 46), CASSFPAYNEQFF (SEQ ID NO: 47), CSVARVQGASGEQYF (SEQ ID NO: 48), CAWVPGTSGRLVF (SEQ ID NO: 49), CASSLGGPSSPLHF (SEQ ID NO: 50), CASSQLDTYNSPLHF (SEQ ID NO: 51), CAWSEGQSSGNTIYF (SEQ ID NO: 52), CASSSQGRAEAFF (SEQ ID NO: 53), CASSLESLNTEAFF (SEQ ID NO: 54), CASSPQRDGYTF (SEQ ID NO: 55), CASSLGGAGTYEQYF (SEQ ID NO: 56), CASSMDRLYSEAFF (SEQ ID NO: 57), CSAMPVNTGELFF (SEQ ID NO: 58), CASSPVLGQVIYGYTF (SEQ ID NO: 59), CASSIGQNYGYTF (SEQ ID NO: 60), CASKGYRERSYNEQFF (SEQ ID NO: 61), CASSYLVGNTEAFF (SEQ ID NO: 62), CSSVKPQGIGTEAFF (SEQ ID NO: 63), CASSPWATSGRTDTQYF (SEQ ID NO: 64), CASSYVGVQPQHF (SEQ ID NO: 65), CASDGGVSYEQYF (SEQ ID NO: 66), CASSFDASRNEQFF (SEQ ID NO: 67), CASSPHDAGADTEAFF (SEQ ID NO: 68), CASSSFWGYNEQFF (SEQ ID NO: 69), CATSDTRAQGYTF (SEQ ID NO: 70), CATSRDLAGAASNQPQHF (SEQ ID NO: 71), CASSLGLAGVLGETQYF (SEQ ID NO: 72), CASSEYLAGVTEQFF (SEQ ID NO: 73), CSASPVLSYEQYF (SEQ ID NO: 74), CASSLSRMPTNYGYTF (SEQ ID NO: 75), CASSPPAGGLTDTQYF (SEQ ID NO: 76), CASSPGTSPYNEQFF (SEQ ID NO: 77), CASSPDRGSSGNTIYF (SEQ ID NO: 78), CASSYGLNYGYTF (SEQ ID NO: 79), CSAKLAPGGELFF (SEQ ID NO: 70), CSARDNRAGGFAEAFF (SEQ ID NO: 81), CASGGAHNSPLHF (SEQ ID NO: 82), CASSGTGYGGPTGELFF (SEQ ID NO: 83), CASRADRGRNTIYF (SEQ ID NO: 84), CASRKRTGSTDTQYF (SEQ ID NO: 85), CASTHWVGNTEAFF (SEQ ID NO: 86), CASSFTRHRGNEKLFF (SEQ ID NO: 87), CASSHNREGYSNTEAFF (SEQ ID NO: 88), CAISWTSGRALINEQYF (SEQ ID NO: 89), CASSLLLRPNTEAFF (SEQ ID NO: 90), CASRGGTENQPQHF (SEQ ID NO: 91), CSVRTGEGQPQHF (SEQ ID NO: 92), CAWWDAYNSPLHF (SEQ ID NO: 93), CASSKWGANYGYTF (SEQ ID NO: 94), CASTTTPNGQGADTQYF (SEQ ID NO: 95), or CAIRITTGDYGYTF (SEQ ID NO: 96).

4. A T cell comprising the TCR of claim 1.

5. The T cell of claim 4, wherein the T cell is a tumor infiltrating lymphocyte (TIL), chimeric antigen receptor (CAR) T cell, or marrow infiltrating lymphocyte (MIL).

6. A method of treating a cancer in a subject comprising administering to the subject a TCR, T cell, CAR T cell, TIL, and/or MIL of claim 1.

7. A method of treating a cancer in a subject comprising administering to the subject a T cell, CAR T cell, TIL, and/or MIL comprising a TCR that recognizes a neoantigen.

8. The method of treating a cancer in a subject of claim 7, wherein the neoantigen recognized by the TCR comprises the sequence DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38).

9. The method of treating a cancer in a subject of claim 7, wherein the TCR comprises the sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), CASSDTARGRNYGYTF (SEQ ID NO: 39), CASSVVGGELFF (SEQ ID NO: 40), CASSPYGTESGANVLTF (SEQ ID NO: 41), CASSQDFRDRAGELFF (SEQ ID NO: 42), CASSQDPSGSYEQYF (SEQ ID NO: 43), CASSQEVGGYTF (SEQ ID NO: 44), CASSQVPGSYEQYF (SEQ ID NO: 45), CSATGTKTNYGYTF (SEQ ID NO: 46), CASSFPAYNEQFF (SEQ ID NO: 47), CSVARVQGASGEQYF (SEQ ID NO: 48), CAWVPGTSGRLVF (SEQ ID NO: 49), CASSLGGPSSPLHF (SEQ ID NO: 50), CASSQLDTYNSPLHF (SEQ ID NO: 51), CAWSEGQSSGNTIYF (SEQ ID NO: 52), CASSSQGRAEAFF (SEQ ID NO: 53), CASSLESLNTEAFF (SEQ ID NO: 54), CASSPQRDGYTF (SEQ ID NO: 55), CASSLGGAGTYEQYF (SEQ ID NO: 56), CASSMDRLYSEAFF (SEQ ID NO: 57), CSAMPVNTGELFF (SEQ ID NO: 58), CASSPVLGQVIYGYTF (SEQ ID NO: 59), CASSIGQNYGYTF (SEQ ID NO: 60), CASKGYRERSYNEQFF (SEQ ID NO: 61), CASSYLVGNTEAFF (SEQ ID NO: 62), CSSVKPQGIGTEAFF (SEQ ID NO: 63), CASSPWATSGRTDTQYF (SEQ ID NO: 64), CASSYVGVQPQHF (SEQ ID NO: 65), CASDGGVSYEQYF (SEQ ID NO: 66), CASSFDASRNEQFF (SEQ ID NO: 67), CASSPHDAGADTEAFF (SEQ ID NO: 68), CASSSFWGYNEQFF (SEQ ID NO: 69), CATSDTRAQGYTF (SEQ ID NO: 70), CATSRDLAGAASNQPQHF (SEQ ID NO: 71), CASSLGLAGVLGETQYF (SEQ ID NO: 72), CASSEYLAGVTEQFF (SEQ ID NO: 73), CSASPVLSYEQYF (SEQ ID NO: 74), CASSLSRMPTNYGYTF (SEQ ID NO: 75), CASSPPAGGLTDTQYF (SEQ ID NO: 76), CASSPGTSPYNEQFF (SEQ ID NO: 77), CASSPDRGSSGNTIYF (SEQ ID NO: 78), CASSYGLNYGYTF (SEQ ID NO: 79), CSAKLAPGGELFF (SEQ ID NO: 70), CSARDNRAGGFAEAFF (SEQ ID NO: 81), CASGGAHNSPLHF (SEQ ID NO: 82), CASSGTGYGGPTGELFF (SEQ ID NO: 83), CASRADRGRNTIYF (SEQ ID NO: 84), CASRKRTGSTDTQYF (SEQ ID NO: 85), CASTHWVGNTEAFF (SEQ ID NO: 86), CASSFTRHRGNEKLFF (SEQ ID NO: 87), CASSHNREGYSNTEAFF (SEQ ID NO: 88), CAISWTSGRALINEQYF (SEQ ID NO: 89), CASSLLLRPNTEAFF (SEQ ID NO: 90), CASRGGTENQPQHF (SEQ ID NO: 91), CSVRTGEGQPQHF (SEQ ID NO: 92), CAWWDAYNSPLHF (SEQ ID NO: 93), CASSKWGANYGYTF (SEQ ID NO: 94), CASTTTPNGQGADTQYF (SEQ ID NO: 95), or CAIRITTGDYGYTF (SEQ ID NO: 96).

10. The method of treating a subject with a cancer of claim 7, wherein the TILs, MILs, T cells, and/or CAR T cells are expanded in vitro in the presence of one or more of the neoantigens prior to administration of the TILs.

11. The method of treating a subject with a cancer of claim 7, further comprising administering to the subject the neoantigen which the T cell, CAR T cell, TIL, and/or MIL recognizes.

12. The method of treating a subject with a cancer of claim 11, wherein the TILs and neoantigen are administered in the same formulation or concurrently.

13. (canceled)

14. (canceled)

15. The method of treating a subject with a cancer of claim 7 further comprising administering to the subject an immune checkpoint inhibitor.

16. The method of treating a subject with a cancer of claim 15, wherein the checkpoint inhibitor comprises a PD1/PDL1 blockade inhibitors and/or CTLA4/B7-1 or 2 inhibitors (such as, for example, PD-1 inhibitors lambrolizumab, OPDIVO® (Nivolumab), KEYTRUDA® (pembrolizumab), and pidilizumab; PD-L1 inhibitors BMS-936559, TECENTRIQ® (Atezolizumab), IMFINZI® (Durvalumab), and BAVENCIO® (Avelumab); and CTLA-4 inhibitors YERVOY (ipilimumab).

17. The method of treating a subject with a cancer of claim 7, wherein the cancer comprises a tyrosine kinase inhibitor resistant or EGFR mutated cancers.

18. The method of treating a subject with a cancer of claim 17, wherein the cancer comprises a mutation, overexpression, activation, inactivation, or fusion of an epithelial growth factor receptor (EGFR) family gene, an anaplastic lymphoma kinase (ALK) gene, a c-ROS oncogene 1 (ROS-1) gene, a MET, a Fibroblast growth factor receptor 1 (FGFR1), B Rapidly Accelerated Fibrosarcoma (BRAF) gene, neurotrophic receptor tyrosing kinase (NTRK) gene, a Rearranged in Transfection (RET) gene, or a Kirsten rat sarcoma viral oncogene homolog (KRAS).

19. The method of treating a subject with a cancer of claim 18, wherein the EGFR family gene comprises EGFR, Human epithelial receptor (HER)-2 (HER-2), HER-3, or HER-4.

20. The method of treating a subject with a cancer of claim 18, wherein the cancer comprises a fusion of ALK and echinoderm microtubule-associated protein-like 4 (EML4), kinesis family member 5B (KIFSB), 5-aminoimidazole-4-carboxamide ribonucleotide transformylase/inosine 5′-monophosphate cyclohydrolase (ATIC), carbamoyl-phosphate synthetase 2, aspartate transcarbamylase, and dihydroorotase (CAD), Clathrin heavy chain 1 (CLTC), Dynactin subunit 1 (DCTN1), Fibronectin (FN1), Huntingtin-interacting protein 1 (HIP1), Kinesin light chain 1 (KLC1), Nucleophosmin (NPM1), Tropomyosin alpha-3 (TPM3), Tropomyosin alpha-4 (TPM4), TFG, Striatin (STRN), Sequestosome-1 (SQSTM1), or RAs-related Nuclear (RAN) binding protein 2 (RANBP2).

21. The method of treating a subject with a cancer of claim 18, wherein the cancer comprises a fusion of RET and kinesis family member 5B (KIF5B), Coiled-coil domain-containing protein 6 (CCDC6), NCOA, EPHA5, PICALM, TRIM33, CUX1, or KIAA1468.

22. The method of treating a subject with a cancer of claim 18, wherein the cancer comprises a fusion of ROS1 and SLC34A2, CD74, SDC4, TPM3, EZR, LRIG3, KDLER2, CCDC6, YWHAE, TFG, or CEP85L.

23. The method of treating a subject with a cancer of claim 7, wherein the cancer comprises non-small cell lung cancer (NSCLC).

24. The method of treating a subject with a cancer of claim 7, further comprising administering to the tyrosine kinase inhibitor resistant cancer crizotinib, ceritinib, alectinib, brigatinib, vemurafenib, dabrafenib, afatinib, Tivantinib, AMG 102, ficlatuzumab, cabozantinib, foretinib, ponatinib, onartuzumab, LKD378, AP26113, TSR-011, Selumetinib, TAE684, Trametinib, barbozatinib, gefitinib, erlotinib, paptinib, vandetanib, afatinib, osimertinib, lenvatinib, nintedanib, pazopanib, regorafenib, sorafenib, sunitinib, bosutinib, dasatinib, imatinib, nilotinib, and/or ponatinib
